(19) United States
(12) Patent Application Publication
(10) Pub. No.: US 2023/0236207 A1
(43) Pub. Date: Jul. 27, 2023

(54) Biomarkers for identifying e-cigarette, or vaping, product use-associated lung injury

(71) Applicant: University of Rochester, Rochester, NY (US)

(72) Inventors: Irfan Rahman, Rochester, NY (US); Matthew McGraw, Rochester, NY (US)

(21) Appl. No.: 18/158,768

(22) Filed: Jan. 24, 2023

Related U.S. Application Data
(60) Provisional application No. 63/303,246, filed on Jan. 26, 2022.

(51) Int. Cl. G01N 33/68; G01N 33/92

(52) U.S. Cl. CPC G01N 33/6893; G01N 33/92; G01N 2800/12

(57) ABSTRACT

The present invention generally relates to novel biomarkers and their methods of use for identifying and treating e-cigarette, or vaping, product use-associated lung injury (EVALI).

### CROSS REFERENCE TO RELATED APPLICATIONS

This application claims priority to U.S. Provisional Application No. 63/303,246, filed Jan. 26, 2022 which is hereby incorporated by reference herein in its entirety.

### STATEMENT REGARDING FEDERALLY SPONSORED RESEARCH OR DEVELOPMENT

This invention was made with government support under HL135613 awarded by the National Institutes of Health. The government has certain rights in the invention.

### BACKGROUND OF THE INVENTION

Electronic cigarettes (e-cigs) or electronic nicotine delivery systems (ENDS), invented in 2003 (see U.S. Pat. No. 8,511,318 B2), were originally conceived as smoking cessation aids but are now a major avenue of nicotine consumption that is rapidly rising in popularity amongst young people (Omaiye E E, et al., Sci Rep, 2019, 9(1):2468). Cannabis e-cigs (CECs) are an adaptation of the same technology used to deliver Δ⁹-tetrahydrocannabinol (Δ⁹-THC) and have become popular among adolescents and young adults (Meehan-Atrash J, et al., RSC Advances, 2021, 11(19):11714-11723). In 2019, the CDC described an outbreak of e-cigarette, or vaping, product use-associated lung injury (EVALI), which as of Feb. 18, 2020, resulted in 2,807 hospitalizations/cases and 68 deaths in the US (cdc.gov/tobacco/basic_information/e-cigarettes/severe-lung-disease.html). The disease presents with symptoms of cough, shortness of breath, chest pain, nausea, vomiting, diarrhea, abdominal pain, fever and chills (Chand H S, et al., Frontiers in Pharmacology, 2020, 10(1619); Kalininskiy A, et al., Pediatr Med, 2021, 4; Adhikari R, et al., Cureus, 2021, 13(2)). The majority of patients/subjects with EVALI have reported the use of THC-based, counterfeit e-cig products (Muthumalage T, et al., Toxics, 2020, 8(3)). In spite of thousands hospitalized with EVALI, there remains a debate on the harmfulness of e-cigs as well as their potential long-term health effects (Song M-A, et al., Cancer Epidemiology, Biomarkers, and prevention, 2020, 29(2):443-451; Marques P, et al., Respir Res, 2021, 22(1):151).

Commonly used as a cutting agent in vaping products, vitamin E acetate (VEA) has been implicated as a key agent in the occurrence of EVALI since its discovery in the bronchoalveolar lavage fluid (BALF) of 48 participants in a study of 51 known lung injury cases (Blount B C, et al., N Engl J Med, 2020, 382(8):697-705). However, VEA was not found in the healthy participants, including 18 e-cig users. Hence, there is some doubt over whether VEA is the sole causative agent. Chemical analyses of illicitly sourced CECs obtained from EVALI subjects demonstrated that these products can contain many different ingredients and adulterants, such as glycerol fatty acid esters, long-chain hydrocarbons, plasticizers, terpenes, metals and more (Duffy B, et al., Toxics, 2020, 8(1); Muthumalage T, et al., Toxics, 2020, 8(2)). Aerosols generated from CECs contain concerning levels of irritants and carcinogens including benzene, isoprene, methyl vinyl ketone, butadiene, toluene, xylenes and more (Meehan-Atrash J, et al., RSC Advances, 2021, 11(19):11714-11723; Meeh-Atrash J, et al., ACS Omega, 2019, 4(14):16111-16120). Muthumalage et al. showed the molecular mechanisms of injurious responses by inhaled illicit cartridges that are shown to cause EVALI (Meehan-Atrash J, et al., RSC Advances, 2021, 11(19):11714-11723). There is also debate on the protective effects of vitamin E acetate against toxicity and vice-versa (Wang S, et al., Toxicology, 2002, 175(1-3):235-245; Hybertson B M, et al., Exp Lung Res, 2005, 31(3):283-294; Meehan-Atrash J and Rahman I, Chem Res Toxicol., 2021, 34(10):2169-2179; Lee H, Medical Hypotheses, 2020, 144). Considering this, it is important to investigate further the mechanisms of EVALI disease development and progression among e-cig users.

Several potential mechanisms have been proposed for the pathogenesis of EVALI (Feldman R, Stanton M, Suelzer E M: Compiling Evidence for EVALI: A Scoping Review of In Vivo Pulmonary Effects After Inhaling Vitamin E or Vitamin E Acetate. J Med Toxicol 2021, 17(3):278-288). In vivo murine inhalation studies using EVALI subject-sourced CECs demonstrate these products can cause cytotoxicity, epithelial barrier dysfunction, and inflammation (Muthumalage T, et al., Toxics, 2020, 8(3)). Mice exposed to EVALI subject-sourced CECs showed increased levels of eicosanoid inflammatory mediators and leukotrienes in BALF, as compared to the mice exposed to VEA or other cutting agents (Muthumalage T, et al., Toxics, 2020, 8(3)). To date, no specific biomarkers for EVALI have been identified and diagnoses are primarily achieved through a process of elimination with the appropriate history of recent e-cigarette use, new physical exam findings, and clinical imaging (Chand H S, et al., Frontiers in Pharmacology, 2020, 10(1619); Kalininskiy A, et al., Pediatr Med, 2021, 4; Adhikari R, et al., Cureus, 2021, 13(2)).

Thus, there is a need in the art for novel biomarkers and improved methods for identifying and treating EVALI. This invention satisfies this unmet need.

### SUMMARY OF THE INVENTION

In one embodiment, the present invention comprises a method of identifying e-cigarette, or vaping, product use associated lung injury (EVALI) in a subject, the method comprising: a) obtaining a biological sample of said subject; b) measuring the level of one or more EVALI-associated biomarkers in said biological sample; c) comparing the level of said one or more EVALI-associated biomarkers in said biological sample to the level of one or more EVALI-associated biomarkers in a comparator; and d) identifying said subject as having EVALI when the level of one or more EVALI-associated biomarker in said biological sample of the subject substantially differs from the level of one or more EVALI-associated biomarker in said comparator.

In one embodiment, said biological sample comprises one or more selected from the group consisting of: whole blood, serum, plasma, urine, and saliva.

In one embodiment, said measuring comprises the use of one or more technique selected from the group consisting of: liquid chromatography with tandem mass spectrometry (LC-MS/MS), enzyme-linked immunosorbent assay (ELISA), bead-based multiplex immunoassay, and polymerase chain reaction.

In one embodiment, said EVALI-associated biomarker comprises one or more molecule selected from the group consisting of: 11-nor-9-carboxy-tetrahydrocannabinol, 8-hydroxy-2′-deoxyguanosine, Prostaglandin E2, and Resolvin D1.

In one embodiment, said EVALI-associated biomarker comprises one or more protein selected from the group consisting of: tumor necrosis factor alpha (TNFα), macrophage inflammatory protein beta (MIP1β), Regulated upon Activation, Normal T Cell Expressed and Presumably Secreted (RANTES), granulocyte-macrophage colony-stimulating factor (GM-CSF), interleukin 9 (IL-9), and club cell protein 10/16 (CC10/16).

In one embodiment, said EVALI-associated biomarker comprises a nucleic acid molecule encoding one or more selected from the group consisting: TNFα, MIP1β, RANTES, GM-CSF, IL-9, and CC10/16.

In one embodiment, said EVALI-associated biomarker comprises a lipid. In one embodiment, the lipid comprises one or more phospholipid selected from the group consisting of: a phosphatidylethanolamine (PE), a phosphatidylcholine, a phosphatidylserine and a phosphoinositol.

In one embodiment, said PE comprises one or more selected from the group consisting of: 1-linoleoyl-2-arachidonoyl-sn-glycero-3-phosphoethanolamine [PE(18:2/20:4)], 1,2-dilinoleoyl-sn-glycero-3-phosphatidylethanolamine [PE(18:2/18:2)], 1-oleoyl-2-linoleyl-sn-glycero-3-phosphoethanolamine [PE(18:1/18:2)], 1-arachidonyl-2-arachidonoyl-sn-glycero-3-phosphoethanolamine [PE(20:0/20:4)], 1-stearoyl-2-eicosapentaenoyl-sn-glycero-3-phosphoethanolamine [PE(18:0/20:5)], PE(P-18:0/20:4), PE(18:0/20:4), PE(18:0/18:2), PE(0:0/18:2), PE(0:0/20:4), PE(16:0/20:4), PE(16:0/20:4), PE(16:0/18:2), PE(16:0/22:6), PE(P-18:0/22:6), PE(0:0/18:1), PE(0:0/18:0), PE(P-16:0/22:6), PE(0:0/16:0), PE(P-16:0/18:2), PE(O-16:0/20:4), PE(18:0/18:1), PE(O-18:0/22:6), PE(18:1/20:4), PE(P-16:0/22:4), PE(0:0/22:6), p-PE(P-18:0/20:5), PE(18:0/22:6), PE(16:0/18:1), PE(P-18:1/22:6), PE(P-18:0/22:4), PE(P-20:0/20:4), PE(P-18:0/18:1), PE(P-16:0/18:1), PE(18:1/18:1), PE(18:0/20:3), p-PE(P-16:0/0:0), PE(0-18:1/0:0), PE(P-18:0/20:3), PE(P-18:0/20:4), PE(P-16:0/20:3), PE(P-16:0/22:6), PE(P-16:0/18:2), PE(P-20:0/18:2), PE(P-20:0/20:5), PE(18:1/22:6), PE(16:0/18:3), PE(0:0/20:3), PE(20:5/18:0), PE(18:0/22:4), PE(P-20:0/20:5), PE(0:0/22:5), PE(P-16:0/20:5), PE(O-16:0/18:2), PE(16:0/22:4), PE(P-18:0/22:6), PE(0:0/18:3), PE(0:0/20:5), PE(16:0/20:5), PE(0:0/16:1), PE(0:0/20:1), and PE(P-20:0/22:4).

In one embodiment, the biomarker comprises acylcarnitine (AC), monounsaturated fatty acid (MUFA), saturated fatty acid (SFA), diglycerides (DG) or fatty acid (FA). In some embodiments, the biomarker comprises determining the sum of acylcarnitine (AC), monounsaturated fatty acid (MUFA), saturated fatty acid (SFA), diglycerides (DG) or fatty acid (FA) in a sample or any combination thereof. In some embodiments, the biomarker comprises determining a ratio of acylcarnitine (AC), monounsaturated fatty acid (MUFA), saturated fatty acid (SFA), diglycerides (DG) or fatty acid (FA) in a sample or any combination thereof.

In some embodiments, the biomarker is the sum of acylcarnitine (AC) in a urine sample. In some embodiments, the sum of ACs in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of monounsaturated fatty acid (MUFA) in a urine sample. In some embodiments, the sum of MUFA in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of saturated fatty acid (SFA) in a urine sample. In some embodiments, the sum of SFA in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of diglycerides (DG) in a urine sample. In some embodiments, the sum of DG in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of fatty acid (FA) in a urine sample. In some embodiments, the sum of FA in a urine sample is decreased in a subject with EVALI as compared to a subject without EVALI (normal or healthy subject) or a comparator control (e.g., the average or normalized ratio of AC:FA in the healthy or normal population).

In some embodiments, the biomarker is the ratio of acylcarnitine (AC) to fatty acid (FA) in a urine sample. In some embodiments, the ratio of AC:FA in a urine sample is elevated in a subject with EVALI as compared to a subject without EVALI (normal or healthy subject) or a comparator control (e.g., the average or normalized ratio of AC:FA in the healthy or normal population).

In one embodiment, the substantial difference comprises a decrease in the level of one or more EVALI-associated biomarker in the subject biological sample as compared to the level of one or more EVALI-associated biomarker in the comparator. In one embodiment, the substantial difference comprises a significantly significant decrease in the level of one or more EVALI-associated biomarker in the subject biological sample as compared to the level of one or more EVALI-associated biomarker in the comparator. For example, in some embodiments, the level of one or more EVALI-associated biomarker in the subject biological sample is statistically significantly decreased as compared to the average level of the biomarker in the same sample type in a population of non-smokers. In some embodiments, the level of one or more EVALI-associated biomarker in the subject biological sample is statistically significantly decreased as compared to the normal range of the level of the biomarker in the same sample type in a population of healthy subjects or a population of non-smokers.

In one embodiment, the substantial difference comprises a decrease of at least 20% in the level of one or more EVALI-associated biomarker in the subject biological sample as compared to the level of one or more EVALI-associated biomarker in the comparator.

In one embodiment, the substantial difference comprises a decrease of at least 30% in the level of one or more EVALI-associated biomarker in the subject biological sample as compared to the level of one or more EVALI-associated biomarker in the comparator.

In one embodiment, the substantial difference comprises an increase in the level of one or more EVALI-associated biomarker in the subject biological sample as compared to the level of one or more EVALI-associated biomarker in the comparator. In one embodiment, the substantial difference comprises a significantly significant increase in the level of one or more EVALI-associated biomarker in the subject biological sample as compared to the level of one or more EVALI-associated biomarker in the comparator. For example, in some embodiments, the level of one or more EVALI-associated biomarker in the subject biological sample is statistically significantly increased as compared to the average level of the biomarker in the same sample type in a population of non-smokers. In some embodiments, the level of one or more EVALI-associated biomarker in the subject biological sample is statistically significantly increased as compared to the normal range of the level of the biomarker in the same sample type in a population of healthy subjects or a population of non-smokers.

In one embodiment, the substantial difference comprises an increase of at least 50% in the level of one or more EVALI-associated biomarker in the subject biological sample as compared to the level of one or more EVALI-associated biomarker in the comparator.

In one embodiment, the substantial difference comprises an increase of at least 80% in the level of one or more EVALI-associated biomarker in the subject biological sample as compared to the level of one or more EVALI-associated biomarker in the comparator.

In one embodiment, said identifying step of the method further comprises confirming the presence one or more additional subject-related parameter selected from the group consisting of: EVALI-associated symptoms, EVALI-associated signs, positive screening for vaping or e-cigarette use within the past 90 days, negative screening for common respiratory infections, and chest imaging results indicative of lung injury.

In one embodiment, said EVALI-associated symptoms comprises one or more selected from the group consisting of: cough, chest tightness, dyspnea, shortness of breath, difficulty breathing, sputum production, pleuritic chest pain, fever, malaise, sweats, chills, myalgias, nausea, abdominal pain, emesis, and diarrhea.

In one embodiment, said EVALI-associated signs comprises one or more selected from the group consisting of: hypoxemia, tachypnea, fever, tachycardia, and hypercarbia.

In one embodiment, said vaping or e-cigarette use within the past 90 days comprises use of one or more selected from the group consisting of: THC-based vaping or e-cigarette products, CBD-based vaping or e-cigarette products, and nicotine-based vaping or e-cigarette products.

In one embodiment, said negative screening for common respiratory infections comprises one or more indicator selected from the group consisting of: no growth of blood, sputum, or urine cultures, and negative respiratory pathogen PCR testing.

In one embodiment, said chest imaging results indicative of lung injury comprises bilateral ground glass opacification of lung imaging fields.

In one embodiment, the method further comprises treating said subject identified as having EVALI with one or more therapy specific to EVALI. In one embodiment, said therapy specific to EVALI comprises administering one or more selected from the group consisting of: oxygen and respiratory/ventilatory support, one or more steroid, one or more exogenous surfactant, one or more anti-inflammatory therapeutic, recombinant CC10/CC16, and exogenous PE with or without surfactant.

In one embodiment, the present invention comprises a method of determining the risk of developing EVALI in a subject, the method comprising: a) obtaining a biological sample of said subject; b) measuring the level of one or more EVALI-associated biomarkers in said biological sample; c) comparing the level of said one or more EVALI-associated biomarkers in said biological sample to the level of one or more EVALI-associated biomarkers in a comparator; and d) determining that said subject is at risk of developing EVALI when the level of one or more EVALI-associated biomarker in said biological sample of the subject substantially differs from the level of one or more EVALI-associated biomarker in said comparator.

In one embodiment, the present invention comprises a method of treating a subject identified as having EVALI, the method comprising: a) obtaining a biological sample of said subject; b) measuring the level of one or more EVALI-associated biomarkers in said biological sample; c) comparing the level of said one or more EVALI-associated biomarkers in said biological sample to the level of one or more EVALI-associated biomarkers in a comparator; d) identifying said subject as having EVALI when the level of one or more EVALI-associated biomarker in said biological sample of the subject substantially differs from the level of one or more EVALI-associated biomarker in said comparator; and e) treating said subject identified as having EVALI with one or more selected from the group consisting of: oxygen and respiratory/ventilatory support, one or more steroid, one or more exogenous surfactant, one or more anti-inflammatory therapeutic, recombinant CC10/CC16, and exogenous PE with or without surfactant.

In one embodiment, the present invention comprises a method of treating a subject who has been identified as having EVALI by detecting a differential level of one or more EVALI-associated biomarkers in a biological sample of the subject as compared to a comparator, wherein the method comprises administering to the subject one or more selected from the group consisting of: oxygen and respiratory/ventilatory support, one or more steroid, one or more exogenous surfactant, one or more anti-inflammatory therapeutic, recombinant CC10/CC16, and exogenous PE with or without surfactant.

In one embodiment, the present invention comprises a method of preparing a biological sample for the detection of one or more EVALI-associated biomarkers to identify EVALI in a subject, the method comprising: a) obtaining a biological sample of said subject; b) subjecting the biological sample to one or more selected from the group consisting of: lipid extraction, one or more anticoagulant, centrifugation, and storage at −80° C.; c) measuring the level of one or more EVALI-associated biomarkers in said biological sample; d) comparing the level of said one or more EVALI-associated biomarkers in said biological sample to the level of one or more EVALI-associated biomarkers in a comparator; and e) identifying said subject as having EVALI when the level of one or more EVALI-associated biomarker in said biological sample of the subject substantially differs from the level of one or more EVALI-associated biomarker in said comparator.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts exemplary results of the flavor preferences of EVALI subjects, comprising self-reported survey data based on favorite flavor (n=6).

FIG. 2 depicts exemplary results demonstrating elevated plasma cotinine and THC levels in EVALI subjects. The level of cotinine (in plasma) and THC metabolites (in urine) from non-users and EVALI subjects was quantified using ELISA. n=6/group. Data are shown as mean±SEM. * p<0.05 as per unpaired t-test.

FIG. 3A and FIG. 3B depict exemplary results of oxidative stress markers in the plasma and urine from EVALI subjects. The levels of myeloperoxidase (MPO) in plasma (FIG. 3A) and 8-isoprostane, and 8-OHdG in urine (FIG. 3B) from non-user and EVALI subjects were measured using ELISA-based assays. n=6/group. Data are shown as mean±SEM. *p<0.05 as per unpaired t-test.

FIG. 4 depicts exemplary changes in the levels of pro-resolving lipid mediators in plasma samples from EVALI subjects. Plasma levels of resolvins (RvD₁, RvD₂ and RvE₁), prostaglandin E₂ (PGE₂), lipoxin B₄ (LXB₄) and thromboxane B2 (TXB2) in non-users and EVALI subjects were quantitated using ELISA-based assays. n=6/group. Data are shown as mean±SEM. * p<0.05, *** p<0.001 as per unpaired t-test.

FIG. 5 depicts exemplary results demonstrating dysregulated levels of inflammatory mediators in plasma from EVALI subjects. Plasma levels of inflammatory cytokines/chemokines in non-users and EVALI subjects were quantitated using Luminex (27-plex) and ELISA (CC10/16)-based assays. n=6/group. Data are shown as mean±SEM. * p<0.05, ** p<0.01 as per unpaired t-test.

FIG. 6 depicts exemplary results demonstrating altered levels of growth factors in subjects with EVALI. Plasma levels of growth factors—VEGF, FGF and PDGF-BB—in non-users and EVALI subjects were determined using Luminex. n=6/group. Data are shown as mean±SEM. P-value calculated per unpaired t-test.

FIGS. 7A through 7C depict exemplary results demonstrating differences in plasma phosphatidylethanolamines (PEs) of the groups studied. FIG. 7A depicts exemplary plasma phosphatidylethanolamine levels in non-users, e-cig users, and EVALI users (n=5 samples/group; median average area with interquartile range reported). FIG. 7B depicts an exemplary volcano plot of lipid species identified comparing non-users and EVALI users (log₂ fold change vs. log₂ p-value). Multiple PEs were identified as significantly reduced (p<0.05). FIG. 7C depicts an exemplary volcano plot of lipid species identified comparing e-cig and EVALI users (log₂ fold change vs. log₂ p-value). Multiple PEs were identified as significantly reduced (p<0.05).

FIG. 8 depicts an exemplary analysis of the sum of urinary acylcarnitine (AC) in EVALI vs. controls.

FIG. 9 depicts an exemplary analysis of the sum of urinary monounsaturated fatty acid (MUFA) in EVALI vs. controls.

FIG. 10 depicts an exemplary analysis of the sum of urinary saturated fatty acid (SFA) in EVALI vs. controls.

FIG. 11 depicts an exemplary analysis of the sum of urinary diglycerides (DG) in EVALI vs. controls.

FIG. 12 depicts an exemplary analysis of the sum of urinary fatty acid (FA) in EVALI vs. controls.

FIG. 13 depicts an exemplary analysis of the ration of urinary AC(sum): FA(sum) in EVALI vs. controls.

### DETAILED DESCRIPTION

The present invention generally relates to novel biomarkers for identifying e-cigarette, or vaping, product use-associated lung injury (EVALI) in subjects, and for methods of using said biomarkers to identify and treat subjects with EVALI. The present invention is based, in part, upon the discovery of biomarkers detected in subject samples with minimally invasive procedures, that differ between EVALI and non-EVALI subjects.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “diagnosis” refers to the determination of the presence of a disease or disorder, such as a bacterial infection. In some embodiments of the present invention, methods for making a diagnosis are provided which permit determination of the presence of a disease or disorder, such as bacterial infection.

As used herein “endogenous” refers to any material from or produced inside an organism, cell, tissue or system.

As used herein “exogenous” refers to any material from or produced outside of an organism, cell, tissue or system.

As used herein, an “immunoassay” refers to any binding assay that uses an antibody capable of binding specifically to a target molecule to detect and quantify the target molecule.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

“Polypeptide”, “peptide” and “protein” as used interchangeably herein, refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “therapy” or “therapeutic regimen” refer to those activities taken to alleviate or alter a disorder or disease, such as EVALI, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

### DESCRIPTION

In some embodiments, the present invention comprises methods of identifying EVALI in a subject. In some embodiments, the method comprises identifying a subject as having EVALI and treating said subject with one or more therapy specific to EVALI. In some embodiments, the method comprises determining the risk of developing EVALI in a subject. In some embodiments, the method comprises evaluating the effectiveness of a treatment for EVALI. In some embodiments, the methods comprise preparing a biological sample for the detection of one or more EVALI-associated biomarkers to identify EVALI in a subject.

### Methods, Assays and Kits for Identification

In some embodiments, the present invention comprises methods of identifying EVALI in a subject, or diagnosing a subject with EVALI. In one embodiment, the method comprises: a) obtaining a biological sample of said subject; b) measuring the level of one or more EVALI-associated biomarkers in said biological sample; c) comparing the level of said one or more EVALI-associated biomarkers in said biological sample to the level of one or more EVALI-associated biomarkers in a comparator; and d) identifying said subject as having EVALI when the level of one or more EVALI-associated biomarker in said biological sample of the subject substantially differs from the level of one or more EVALI-associated biomarker in said comparator. For example, in some embodiments, the subject is diagnosed with EVALI when the level of the biomarker is increased in the sample of the subject as compared to the average or normalized level of the biomarker in one or more normal subjects. In some embodiments, the subject is diagnosed with EVALI when the level of the biomarker is decreased in the sample of the subject as compared to the average or normalized level of the biomarker in one or more normal subjects.

A skilled artisan will recognize that the biological sample may be of any biological tissue or fluid. The biological sample can be a sample from any source which contains, or may contain, one or more of the described biomarkers, such as a body fluid (e.g., blood, plasma, serum, urine etc.), or a tissue, or a tumor, or a cell, or a combination thereof. A biological sample can be obtained by appropriate methods, such as, by way of examples, biopsy or fluid draw. In some embodiments, the biological sample can be processed to enhance access to lipids, polypeptides, nucleic acids, and the processed biological sample can then be used in the described methods.

Exemplary biological samples include, but are not limited to, blood, saliva, mucous, sputum, bronchoalveolar fluid (BALF) or BAL cells, urine, milk, and semen. In some embodiments, the blood sample comprises whole blood, serum or plasma.

A skilled artisan will further recognize that any methods of obtaining a biological sample known in that art can be used with the methods of the present invention. In one embodiment, the sample is collected via one or more minimally invasive method. In one embodiment, the sample is collected via a blood draw. In one embodiment, the sample is collected via saliva collection. In one embodiment, the sample is collected via urine collection.

The biomarkers of the present invention can be any biomarkers associated with EVALI. Exemplary biomarkers include, but are not limited to, proteins, nucleic acid molecules, lipids, metabolites, antibodies, inflammatory mediators, lipid mediators, and oxidative stress markers.

In some embodiments, the biomarker comprises one or more metabolite of tetrahydrocannabinol (THC) or nicotine. In some embodiments, the biomarker comprises a metabolite of Δ⁸, Δ⁹ or Δ¹⁰ or →^{o} THC. In one embodiment, the metabolite of THC comprises (−)-11-nor-9-carboxy-Δ⁹-THC. In one embodiment, the metabolite of nicotine comprises cotinine. In one embodiment, the biomarker comprises (−)-11-nor-9-carboxy-Δ⁹-THC.

In some embodiments, the biomarker comprises one or more marker of oxidative stress. Exemplary markers of oxidative stress include, but are not limited to, 8-Isoprostane and 8-hydroxy-2′-deoxyguanosine (8-OHdG). In one embodiment, the biomarker comprises 8-OHdG.

In some embodiments, the biomarker comprises one or more lipid mediator. Exemplary lipid mediators include, but are not limited to, RvD₁, RvD₂, RvE₁, LXB₄ and PGE₂. In one embodiment, the biomarker comprises RvD₁. In one embodiment, the biomarker comprises PGE₂.

In some embodiments, the biomarker comprises one or more inflammatory mediator. Exemplary inflammatory mediators include, but are not limited to, TNFα, IL-1β, MCP-1, IL-8, IL-6, CC10/16, MIP1β, RANTES, GM-CSF, Basic FGF, VEGF, PDGF-BB, IL-1rα, IL-2, IL-4, IL-5, IL-7, IL-9, IL-10, IL-13, IL-15, IL-17, Eotaxin, IFNγ, G-CSF, IL-12p70, MIP-1α, and IP10. In one embodiment, the biomarker is selected fromt the group consisting of: tumor necrosis factor alpha (TNFα), macrophage inflammatory protein beta (MIP1β), Regulated upon Activation, Normal T Cell Expressed and Presumably Secreted (RANTES), granulocyte-macrophage colony-stimulating factor (GM-CSF), interleukin 9 (IL-9), and club cell protein 10/16 (CC10/16).

In some embodiments, the biomarker comprises one or more nucleic acid molecule encoding one or more inflammatory mediator. Exemplary inflammatory mediators are described. In one embodiment, the biomarker comprises a nucleic acid molecule encoding one or more selected from the group consisting: TNFα, MIP1β, RANTES, GM-CSF, IL-9, and CC10/16.

In some embodiments, the biomarker comprises one or more lipid. Exemplary lipids include, but are limited to, acidic glycosphingolipids, ceramides, diacylglycerols, fatty acids and conjugates, glycerophosphocholines, glycerophosphoethanolamines, glycerophosphoglycerols, glycerophosphoinositols, glycerophosphoserines, monoradylglycerols, neutral glycosphingolipids, sphingomyelins, sterols, and triacylglycerols.

In one embodiment, the biomarker comprises one or more phospholipid. In some embodiments, the phospholipid is selected from the group consisting of: a phosphatidylethanolamine (PE), a phosphatidylcholine (PC), a phosphatidylserine (PS) and a phosphoinositol (PI).

In one embodiment, the biomarker comprises one or more PE. Exemplary PEs include, but are not limited to, PE(18:2/20:4), PE(18:2/18:2), PE(18:1/18:2), PE(20:0/20:4), PE(18:0/20:5), PE(P-18:0/20:4), PE(18:0/20:4), PE(18:0/18:2), PE(0:0/18:2), PE(0:0/20:4), PE(16:0/20:4), PE(16:0/20:4), PE(16:0/18:2), PE(16:0/22:6), PE(P-18:0/22:6), PE(0:0/18:1), PE(0:0/18:0), PE(P-16:0/22:6), PE(0:0/16:0), PE(P-16:0/18:2), PE(O-16:0/20:4), PE(18:0/18:1), PE(O-18:0/22:6), PE(18:1/20:4), PE(P-16:0/22:4), PE(0:0/22:6), p-PE(P-18:0/20:5), PE(18:0/22:6), PE(16:0/18:1), PE(P-18:1/22:6), PE(P-18:0/22:4), PE(P-20:0/20:4), PE(P-18:0/18:1), PE(P-16:0/18:1), PE(18:1/18:1), PE(18:0/20:3), p-PE(P-16:0/0:0), PE(O-18:1/0:0), PE(P-18:0/20:3), PE(P-18:0/20:4), PE(P-16:0/20:3), PE(P-16:0/22:6), PE(P-16:0/18:2), PE(P-20:0/18:2), PE(P-20:0/20:5), PE(18:1/22:6), PE(16:0/18:3), PE(0:0/20:3), PE(20:5/18:0), PE(18:0/22:4), PE(P-20:0/20:5), PE(0:0/22:5), PE(P-16:0/20:5), PE(O-16:0/18:2), PE(16:0/22:4), PE(P-18:0/22:6), PE(0:0/18:3), PE(0:0/20:5), PE(16:0/20:5), PE(0:0/16:1), PE(0:0/20:1), and PE(P-20:0/22:4).

In one embodiment, the biomarker comprises acylcarnitine (AC), monounsaturated fatty acid (MUFA), saturated fatty acid (SFA), diglycerides (DG) or fatty acid (FA). In some embodiments, the biomarker comprises determining the sum of acylcarnitine (AC), monounsaturated fatty acid (MUFA), saturated fatty acid (SFA), diglycerides (DG) or fatty acid (FA) in a sample or any combination thereof. In some embodiments, the biomarker comprises determining a ratio of acylcarnitine (AC), monounsaturated fatty acid (MUFA), saturated fatty acid (SFA), diglycerides (DG) or fatty acid (FA) in a sample or any combination thereof.

In some embodiments, the biomarker is the sum of acylcarnitine (AC) in a urine sample. In some embodiments, the sum of ACs in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of monounsaturated fatty acid (MUFA) in a urine sample. In some embodiments, the sum of MUFA in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of saturated fatty acid (SFA) in a urine sample. In some embodiments, the sum of SFA in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of diglycerides (DG) in a urine sample. In some embodiments, the sum of DG in a urine sample is elevated in a subject with EVALI as compared to a subject without EVALI (normal or healthy subject) or a comparator control (e.g., the average or normalized ratio of AC:FA in the healthy or normal population).

In some embodiments, the biomarker is the sum of fatty acid (FA) in a urine sample. In some embodiments, the sum of FA in a urine sample is decreased in a subject with EVALI as compared to a subject without EVALI (normal or healthy subject) or a comparator control (e.g., the average or normalized ratio of AC:FA in the healthy or normal population).

In some embodiments, the biomarker is the ratio of acylcarnitine (AC) to fatty acid (FA) in a urine sample. In some embodiments, the ratio of AC:FA in a urine sample is elevated in a subject with EVALI as compared to a subject without EVALI (normal or healthy subject) or a comparator control (e.g., the average or normalized ratio of AC:FA in the healthy or normal population).

One skilled in the art will recognize and understand the standardized notations used to differentiate lipids identified by mass spectrometry. (See Liebisch et al., J Lipid Res, 2013 June, 54(6): 1523-1530; incorporated herein by reference in its entirety).

One of skill in the art will further recognize that any methods of measuring one or more biomarkers can be used in the methods of present invention. Exemplary methods for measuring biomarkers include, but are not limited to, chromatography (e.g., liquid chromatography), spectrometry (e.g., mass spectrometry), immunoassays, enzymatic assays, colorimetric assays, and nucleic acid assays (e.g, identification and/or amplification assays). Further non-limiting examples include microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), pyrolysis mass spectrometry, nephelometry, dispersive Raman spectroscopy, gas chromatography, gas chromatography combined with mass spectrometry, matrix-assisted laser desorption ionization-time of flight (MALDI-TOF) combined with mass spectrometry, ion spray spectroscopy combined with mass spectrometry, capillary electrophoresis, and surface plasmon resonance (such as according to systems provided by Biacore Life Sciences). See also PCT Publications WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Methods of detecting and/or quantifying protein levels in a biological sample of a subject include, but are not limited to, an immunochromatography assay, an immunodot assay, a bead-based microarray (e.g., Luminex) assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (MA), a radioimmunodiffusion assay, an ouchterlony immunodiffusion assay, reverse phase protein microarray, a rocket immunoelectrophoresis assay, an immunohistostaining assay, an immunoprecipitation assay, a complement fixation assay, flow cytometry, FACS, an enzyme-substrate binding assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

Methods for detecting and/or quantifying a nucleic acid (e.g., mRNA), such as RT-PCR, real time PCR, microarray, branch DNA, NASBA and others, are well known in the art. Using sequence information provided by the database entries for the biomarker sequences, expression of the biomarker sequences can be detected (if present) and measured using techniques well known to one of ordinary skill in the art. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., TMA, SDA, NASBA), signal amplification methods (e.g., bDNA), nuclease protection assays, in situ hybridization and the like.

Methods for detecting and/or quantifying lipids include, but are not limited, to thin-layer chromatography combined with gas chromatography, nuclear magnetic resonance spectroscopy (1H-NMR), mass spectroscopy (MS). Lipid identification and quantification is facilitated by advances in mass spectrometry and lipid biochemistry, which enable the simultaneous high throughput identification and quantification of hundreds of molecular lipid species in several lipid classes collectively referred to as the lipidome. Lipidomic studies identify lipid cellular distribution and describe their biochemical mechanisms, interactions and dynamics. Importantly, lipidomics quantifies the exact chemical composition of lipidomes. The identification of molecular lipid species (e.g., PC 16:0/18:1) is the main feature of advanced lipidomics, which delivers highly resolved molecular lipid species rather than summed fatty acid information. For example, the information of the type of fatty acids and their positions of attachment to the glycerol backbone making up the particular PC molecule is revealed.

Methods for detecting and/or quantifying biomolecules (e.g., metabolites) are well known in the art and include, but are not limited to, mass spectrometry with or without prior separation (e.g., gas chromatography, liquid chromatography, capillary electrophoresis, ion mobility, etc.), immunoassays (e.g., immunoblotting assays, ELISAs, radioimmunoassays, electrochemiluminescence-based detection assays, magnetic immunoassays, lateral flow assays, etc.), and enzyme-coupled assays.

In one embodiment, the method of measuring comprises one or more selected from the group consisting of: liquid chromatography with tandem mass spectrometry (LC-MS/MS), enzyme-linked immunosorbent assay (ELISA), bead-based multiplex immunoassay, and polymerase chain reaction.

In one embodiment, the method of the present invention comprises comparing the level of one or more EVALI-associated biomarkers in a biological sample to the level of one or more EVALI-associated biomarkers in a comparator. The comparator can comprise a positive control, negative control, a historical control, a historical norm, a wild-type control, a baseline level or a reference value. The comparator may comprise a biological sample from a subject that does not have EVALI, a biological sample from a subject that does not have a history of vaping or e-cigarette use (e.g., within the past 90 days), and/or a biological sample from the same subject prior to developing one or more symptom and/or sign of EVALI.

In one embodiment, the method of the present invention comprises identifying a subject as having EVALI when the level of one or more EVALI-associated biomarker in a biological sample of the subject substantially differs from the level of one or more EVALI-associated biomarker in a comparator. In some embodiments, said identifying comprises diagnosing said subject with EVALI. In some embodiments, said identifying further comprises providing a prognosis.

A “substantial difference”, as used herein, can refer to an increase or decrease of one or more biomarker in the subject sample as compared to the comparator. In some embodiments, the substantial difference is a statistically significant difference.

In some embodiments, the difference is determined to be substantial when the biomarker of the subject sample is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator.

In some embodiments, the difference is determined to be substantial when the biomarker of the subject sample is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator.

In some embodiments, the biomarker comprising (−)-11-nor-9-carboxy-Δ⁹-THC is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator. In one embodiment, the biomarker comprising (−)-11-nor-9-carboxy-Δ⁹-THC is increased by at least about 190%.

In some embodiments, the biomarker comprising 8-OHdG is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator. In one embodiment, the biomarker comprising 8-OHdG is increased by at least about 80%.

In some embodiments, the biomarker comprising RvD₁ is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator. In one embodiment, the biomarker comprising RvD₁ is decreased by at least about 70%.

In one embodiment, the biomarker comprising PGE₂ is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator. In one embodiment, the biomarker comprising PGE₂ is decreased by at least about 85%.

In some embodiments, the biomarker comprising TNFα, MIP1β, RANTES, GM-CSF, IL-9, or CC10/16 and/or a nucleic acid encoding TNFα, MIP1β, RANTES, GM-CSF, IL-9, or CC10/16 is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator.

In one embodiment, the biomarker comprising TNFα is decreased by at least about 60%. In one embodiment, the biomarker comprising MIP1β is decreased by at least about 20%. In one embodiment, the biomarker comprising RANTES is decreased by at least about 65%. In one embodiment, the biomarker comprising GM-CSF is decreased by at least about 75%. In one embodiment, the biomarker comprising IL-9 is decreased by at least about 30%. In one embodiment, the biomarker comprising CC10/16 is decreased by at least about 55%.

In some embodiments, the biomarker comprising one or more phospholipid is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator.

In some embodiments, the biomarker comprising one or more phosphatidylethanolamine is increased or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000% in the subject sample relative to the comparator. In one embodiment, the biomarker comprising one or more phosphatidylethanolamine is decreased by at least about 30%.

In some embodiments, said identifying of the method further comprises confirming the present of one or more additional subject-related parameter associated with EVALI. By way of example, EVALI-associated symptoms, EVALI-associated signs, positive screening for vaping or e-cigarette use within the past 90 days, negative screening for common respiratory infections, and chest imaging results indicative of lung injury, represent a non-limiting list of parameters that can be used in combination with the method of the present invention to confirm EVALI.

Non-limiting examples of EVALI-associated symptoms include cough, chest tightness, dyspnea, shortness of breath, difficulty breathing, sputum production, pleuritic chest pain, fever, malaise, sweats, chills, myalgias, nausea, abdominal pain, emesis, and diarrhea. Non-limiting examples of EVALI-associated signs include hypoxemia, tachypnea, fever, tachycardia, and hypercarbia.

In some embodiments, positive screening for vaping or e-cigarette use within the past 90 days comprises screening for use of THC-based vaping or e-cigarette products, CBD-based vaping or e-cigarette products, and/or nicotine-based vaping or e-cigarette products.

In some embodiments, negative screening for common respiratory infections includes, but is not limited to, no detectable growth of blood, sputum, or urine cultures, and negative respiratory pathogen PCR testing.

In one embodiment, the chest imaging results indicative of lung injury include, but is not limited to, bilateral ground glass opacification of lung imaging fields.

In some embodiments, the method comprises determining the risk of developing EVALI in a subject. In one embodiment, the method comprises: a) obtaining a biological sample of the subject; b) measuring the level of one or more EVALI-associated biomarkers in said biological sample; c) comparing the level of said one or more EVALI-associated biomarkers in said biological sample to the level of one or more EVALI-associated biomarkers in a comparator; and d) determining that said subject is at risk of developing EVALI when the level of one or more EVALI-associated biomarker in said biological sample of the subject substantially differs from the level of one or more EVALI-associated biomarker in said comparator.

In some embodiments, the present invention comprises an assay for detecting a difference between the level of one or more biomarkers associated with EVALI in biological sample of a subject and the level of one or more biomarkers associated with EVALI in a comparator. In some embodiments, the present invention comprises an assay for diagnosing EVALI, comprising means for detecting a difference between the level of one or more biomarkers associated with EVALI in biological sample of a subject and the level of one or more biomarkers associated with EVALI in a comparator.

The present invention further comprises kits useful for performing the methods of the present invention. In one embodiment, the kit comprises means for detecting a difference between the level of one or more biomarkers associated with EVALI in biological sample of a subject and the level of one or more biomarkers associated with EVALI in a comparator.

Such kits comprise various combinations of components useful in any of the methods described elsewhere herein, including for example, materials for quantitatively analyzing a biomarker of the invention (e.g., a lipid, protein, and/or nucleic acid), and instructional material. For example, in one embodiment, the kit comprises components useful for the quantification of a desired nucleic acid in a biological sample. In another embodiment, the kit comprises components useful for the quantification of a desired protein in a biological sample. In one embodiment, the kit comprises components useful for the quantification of a desired lipid in a biological sample. In one embodiment, the kit comprises components useful for the quantification of a desired biological molecule in a biological sample.

In various embodiments, to determine whether the level of a biomarker of the invention is increased or decreased in a biological sample obtained from the subject, the level of the biomarker is compared with the level of at least one comparator control contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

Methods of Treatment

In some embodiments, the methods of the invention further comprise treating a subject identified as having EVALI with one or more therapy specific to EVALI. In one embodiment, the method comprises: a) obtaining a biological sample of a subject; b) measuring the level of one or more EVALI-associated biomarkers in said biological sample; c) comparing the level of said one or more EVALI-associated biomarkers in said biological sample to the level of one or more EVALI-associated biomarkers in a comparator; and d) identifying said subject as having EVALI when the level of one or more EVALI-associated biomarker in said biological sample of the subject substantially differs from the level of one or more EVALI-associated biomarker in said comparator; and e) treating said subject identified as having EVALI with one or more therapy specific to EVALI.

In some embodiments, the method comprises evaluating the effectiveness of a treatment for EVALI in a subject having EVALI. In one embodiment, the method comprises: a) obtaining a first biological sample of a subject having EVALI and measuring the level of one or more EVALI-associated biomarkers in said first biological sample; b) administering to the subject one or more therapy to treat EVALI; c) obtaining a second biological sample of the subject having EVALI and measuring the level of one or more EVALI-associated biomarkers in said second biological sample; d) comparing the level of said one or more EVALI-associated biomarkers in said first biological sample to the level of one or more EVALI-associated biomarkers in said second biological sample; and e) determining that said therapy to treat EVALI is effective when the level of one or more EVALI-associated biomarker in said second biological sample of the subject substantially differs from the level of one or more EVALI-associated biomarker in said first biological sample. In some embodiments, the substantial difference comprises an increase. In some embodiments, the substantial difference comprises a decrease.

In one embodiment, the method of evaluating the effectiveness of a treatment for EVALI in a subject having EVALI comprises: a) administering to the subject having EVALI one or more therapy to treat EVALI; b) obtaining a biological sample of the subject and measuring the level of one or more EVALI-associated biomarkers in said biological sample; c) comparing the level of said one or more EVALI-associated biomarkers in said biological sample to the level of one or more EVALI-associated biomarkers in a comparator; and d) determining that said therapy to treat EVALI is effective when the level of one or more EVALI-associated biomarker in said biological sample of the subject does not substantially differ from the level of one or more EVALI-associated biomarker in the comparator. In some embodiments, the comparator comprises a reference control (e.g., a biological sample of a subject that does not have EVALI).

In some embodiments, the method comprises treating a subject who has been identified as having EVALI by detecting a differential level of one or more EVALI-associated biomarkers in a biological sample of the subject as compared to a comparator, wherein the method comprises administering to the subject one or more therapy specific to EVALI.

Exemplary therapies for treating EVALI (i.e., specific to EVALI) include, but are not limited to, oxygen and respiratory/ventilatory support, one or more steroid, one or more exogenous surfactant, one or more anti-inflammatory therapeutic, recombinant CC10/CC16, and exogenous PE with or without surfactant. A skilled artisan will recognize, however, that therapies for treating EVALI are not limited to those described herein.

Steroids of the present invention can be delivered via any route suitable treating EVALI including, but not limited to, topical and systemic delivery. Non-limiting examples of steroids include Prednisone, Prednisolone, Methylprednisolone, Beclomethasone, Betamethasone, Dexamethasone, Hydrocortisone, Triamcinolone, Fluticasone, Mometasone, Ciclesonide, Flunisolide, and Budesonide.

Non-limiting example of exogenous surfactants include beractant, lucinactant, calfactant, poractant, colfosceril palmitate, and pumactant.

Non-limiting examples of anti-inflammatory therapeutics include ibuprofen, naproxen, diclofenac, celecoxib, mefenamic acid, etoricoxib, indomethacin, high-dose aspirin, and recombinant club cell secretory protein (rhCCSP).

Methods of Preparation

In some embodiments, the present invention comprises a method of preparing a biological sample for the detection of one or more EVALI-associated biomarkers to identify EVALI in a subject. In one embodiment, the method comprises: a) obtaining a biological sample of a subject; b) subjecting the biological sample to one or more method that transforms the biological sample into a form suitable for measurement; c) measuring the level of one or more EVALI-associated biomarkers in said biological sample; d) comparing the level of said one or more EVALI-associated biomarkers in said biological sample to the level of one or more EVALI-associated biomarkers in a comparator; and e) identifying said subject as having EVALI when the level of one or more EVALI-associated biomarker in said biological sample of the subject substantially differs from the level of one or more EVALI-associated biomarker in said comparator.

One skilled in the art will recognize that any known methods of preparing a sample for measurement of one or more biomarker can be used in the methods of the present invention. Exemplary methods include, but are not limited to, extraction, centrifugation, isolation, and cryopreservation. In one embodiment, the method of preparing the biological sample is selected from the group consisting of: lipid extraction, protein extraction, nucleic acid isolation, contacting with one or more anticoagulant, centrifugation, and cryopreservation.

Any known methods of lipid extraction can be used with the methods of the present invention. In some embodiments, the lipid extraction is such that is suitable for measuring lipids in the sample by mass spectrometry. In some embodiments, the lipid extraction is such that is suitable for measuring lipids by liquid chromatography with tandem mass spectrometry (LC-MS/MS).

By way of example, lipid extraction may be performed as follows: For total lipid extraction, a methyl-tert-butyl ether (MTBE)-based liquid-liquid extraction may be used: 520 μL methanol containing 20 μL of an internal standard mixture, 480 μL methanol, and 4 μL MTBE may be sequentially added to each sample. After vigorous vortexing, the mixture may be incubated on a tabletop shaker at 550 rpm at room temperature for 1 h. Phase separation may be induced by the addition of 1 mL water. The samples may be shaken for 10 min, then centrifuged at 2000×g for 20 min. The upper organic phase of each sample may be carefully removed using a Pasteur pipette and transferred into a clean glass tube. The remaining aqueous phase may be reextracted with 2.5 mL of MTBE/methanol/water 10:3:2.5 (v/v/v). After vortexing and centrifugation as above, the organic phase may be collected and combined with the initial organic phase. The extracted lipids may then be dried in a SpeedVac vacuum concentrator. Finally, the dried lipid extracts may be reconstituted in 200 μL n-butanol/methanol 1:1 (v/v) and transferred into autosampler vials for analysis by LC-MS/MS.

Any known methods of nucleic acid isolation suitable for measuring the nucleic acid molecule may be used with the methods of the present invention. In some embodiments, the nucleic acid isolation is such that is suitable for amplifying nucleic acid (e.g, via PCR). In some embodiments, the nucleic acid isolation is such that is suitable for detecting the nucleic acid. In In some embodiments, the nucleic acid isolation is such that is suitable for sequencing the nucleic acid. In some embodiments, the nucleic acid isolation is such that is suitable for quantifying the nucleic acid. A skilled artisan will recognize that there are numerous commercially available kits and publicly accessible protocols for nucleic acid isolation.

Any known methods of protein extraction may be used with the methods of the present invention. In some embodiments, the protein extraction is such that is suitable for detecting the protein via one or more immunoassay (e.g., Western Blot). In some embodiments, the protein extraction is such that is suitable for quantifying the protein via one or more immunoassay (e.g., ELISA). A skilled artisan will recognize that there are numerous commercially available kits and publicly accessible protocols for protein extraction.

Any known methods of processing a blood sample can be used with the methods of the present invention. In some embodiments, a blood sample is contacted with one or more anticoagulant concurrent with or subsequent to the blood being drawn. Exemplary anticoagulants include, but are not limited to, ethylenediaminetetraacetic acid (EDTA) salts, heparin salts, citrate salts, oxalate salts, and acid citrate dextrose (ACD). In one embodiment, the blood sample is subject to centrifugation to remove blood cells.

Any known methods of cryopreservation can be used with the methods of the present invention. In some embodiments, the sample is cryopreserved at a temperature of at least at least −50, at least −60, at least −70, at least −80, at least −90, at least −100, at least −110, at least −120, at least −130, at least −140, at least −150, at least −160, at least −170, at least −180, at least −190, or at least −200 degrees Celsius.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Non-Invasive Systemic Biomarkers of EVALI

Systemic biomarkers of oxidative stress, inflammation, and lipid mediators have been studied in smokers (Liu et al., 2011, Cancer Epidemiol Biomarkers Prev, 20(8):1760-1769; Shiels et al. 2014, J Natl Cancer Inst, 106(11); Rahman et al., 1996, Am J Respir Crit Care Med, 154(4 Pt 1):1055-1060), COPD patients (Stockley et al., Am J Respir Crit Care Med 2019, 199(10):1195-1204; Garcia-Rio et al., Respir Res 2010, 11:63; Selvarajah et al., Mediators Inflamm 2016, 2016:3604842) and ENDS users (Muthumalage et al., Toxics 2020, 8(3); Song et al., Cancer Epidemiology, Biomarkers, and prevention 2020, 29(2):443-451; Singh et al., ERJ Open Res 2019, 5(4)). However, most studies at best can only imply and extrapolate to EVALI clinical cases (Muthumalage et al., Toxics 2020, 8(3); Singh et al., ERJ Open Res 2019, 5(4); McDonough et al., Am J Physiol Lung Cell Mol Physiol 2021, 320(5):L661-L679; Guerrini et al., The Lancet Respiratory Medicine 2020, 8(2); Mokra et al., Respiratory Physiology & Neurobiology 2015, 209:52-58). Little is known to assess potential biomarkers of EVALI. While attempting to bridge this gap in research literature, the aim of the present study was to identify biomarkers of oxidative stress, lipid mediators, and inflammatory responses that may play a role in the pathogenesis of EVALI.

### Methods

Participants. Participants were recruited through various local newspapers, magazine advertisements, and flyers along with word of mouth (IRB approval #CR00003968), or at the time of admission during initial diagnosis for EVALI. Subjects in the EVALI group met clinical criteria with: (1) recent use (within 90 days of hospitalization) of e-cigarette, or vaping, products, (2) bilateral ground-glass opacities on radiographic imaging, and (3) exclusion of other common causes for changes on chest imaging, including community-acquired pneumonia (Kalininskiy et al., Lancet Respiratory Medicine 2019, 7(12):1017-1026; Navon et al., Morbidity and Mortality Weekly Report (MMWR) 2019, 68(45):1034-1039). The majority (5 of 6) of samples from EVALI subjects were obtained prior to corticosteroid treatment. Participants were selected based on a self-reported questionnaire. To be eligible, all participants had to be between the ages of 18 and 35 years. Additionally, all participants were screened for a history of chronic illness such as heart and lung disease, diabetes, cancer, and/or current viral flu/pneumonia infections. For non-users, participants also had to disclose if they were currently taking any anti-inflammatories or corticosteroid drugs and if so, were removed from the study. Participants were excluded if pregnant or breastfeeding. Participants in the non-user group were required to never have smoked any tobacco products. Participants in the EVALI group were screened for history of e-cigarette use/vaping in addition to their diagnosis. Written informed consent was obtained from all study participants. See Tables 1 and 2 below for the clinical presentation of EVALI subjects and their median peak laboratory values.

TABLE 1
Clinical Presentation of EVALI subjects (n = 6)

| Chief Complaint | |
|---|---|
| Gastrointestinal (GI) | 3/6 (60%) |
| Respiratory | 2/6 (33%) |
| Combination | 3/6 (50%) |
| Respiratory support | |
| Positive pressure ventilation | 1/6 (17%) |
| Supplemental oxygen | 5/6 (83%) |
| No respiratory support | 1/6 (17%) |
| Antibiotics | 6/6 (100%) |
| Median duration of hospitalization (days, IQR) | 5.5 (3.0-6.8) |
| Median duration of system steroids (days, IQR) | 9.0 (6.5-15.7) |

TABLE 2
Median peak laboratory values for EVALI subjects (n = 6)

| Lab Value | Median (IQR) | Reference Units | |
|---|---|---|---|
| C-Reactive Protein (CRP) | 332.5 (210.0-390.3) | <5.0 | mg/L |
| Estimated Sedimentation | 71.5 (39.0-95.3) | 0-20 | mm/hr |
| Rate (ESR) | | | |
| Total WBC count | 15.3 (12.4-18.5) | 3.8-10.5 | K/μl |
| Neutrophils percentage | 91.5 (89.8-92.3) | 40-60% | |
| Neutrophil number | 14.0 (11.1-17.1) | 2.5-7.0 | K/μl |

TABLE 3
Demographic and vaping status characteristics of non-users and
EVALI patients; N/A: not applicable.

| | Non-users | EVALI patients |
|---|---|---|
| | (n = 6) | (n = 6) |
| Age, years | | |
| Mean (SD) | 22 (2.44) | 22.3 (6.4) |
| Range | 18-25 | 19-35 |
| Sex, n (%) | | |
| Male | 3 (50) | 3 (50) |
| Female | 3 (50) | 3 (50) |
| Demographics, n | | |
| Caucasian | 5 | 3 |
| African American | 1 | 2 |
| Asian | 0 | 0 |
| Not Hispanic/Latino/Black (Not White) | 0 | 1 |
| E-cig use, n (%) | | |
| Vaping Frequency | N/A | |
| 10+ times/day | | 3 (50%) |
| 3-5 times/day | | 2 (33.3%) |
| <1 time/day | | 1 (16.6%) |
| Duration of Vape per Session | N/A | |
| 20+ min | | 0 |
| 10-14 min | | 2 (33.3%) |
| 5-9 min | | 0 |
| <5 min | | 4 (66.6%) |
| Vaping years | N/A | |
| >5+ yr. | | 0 |
| 2-5 yr. | | 2 (33.3) |
| 1-2 yr. | | 2 (33.3) |
| < l yr. | | 2 (33.3) |
| Hospitalizations, n (%) | 0 | 6 (100) |

Demographics. During the questionnaire, participants provided their age, sex, and ethnicity. E-cigarette use duration, e-cigarette duration per session, e-cigarette smoking frequency, flavor type and approximate amount of nicotine (high/low) in each flavor were also disclosed. The participants were categorized into two groups: Non-user and EVALI subjects (see Table 3).

Sample Collection. Whole venous blood (approximately 20-25 mL) was collected from participants in vacutainer tubes containing EDTA and spun at 1000 g for 10 min to obtain plasma and stored immediately at −80° C. until further use. Participants provided urine and samples were immediately stored at −80° C. for further use.

Measurement of Biomarkers by Multiplex Panel Assay. Cytokine/mediator levels in plasma from non-users and EVALI subjects were quantified by Bio-Plex Pro Human 27-Plex assay (M500KCAF0Y, Bio-Rad, Hercules, Calif.) assay with a 1:4 sample dilution per manufacturer's instructions on a FLEXMAP 3D instrument (Luminex, Austin, Tex.).

Measurement of Biomarkers by ELISA/EIA. Commercially available kits were used for quantifying Resolvin D₁ (Cat #500380, Cayman Chemical, Ann Arbor, Mich.), Resolvin D₂ (Cat #501120, Cayman Chemical, Ann Arbor, Mich.), Resolvin E₁ (Cat #MBS286046, MyBioSource, San Diego, Calif.), Lipoxin B₄(Cat #MBS9380211, MyBioSource, San Diego, Calif.), Prostaglandin E₂ (Cat #514010, Cayman Chemical, Ann Arbor, Mich.), Cotinine (Cat #1-2002, Salimetrics, Carlsbad, Calif.), and CC10/16 (Cat #DUGB00, R&D Systems, Minneapolis, Minn.) in plasma. In urine samples, 8-OHdG (Cat #4380-096K, R&D systems, Minneapolis, Minn.), 8-Isoprostane (Cat #516351 Cayman Chemical, Ann Arbor, Mich.) and THC metabolite, predominantly 11-nor-9-carboxy-Δ⁹-THC i.e. Δ⁹-THC, (Cat #701570 Cayman Chemical, Ann Arbor, Mich.) were run according to the manufacturer's directions, respectively.

Statistical Analysis. Data from all assays were analyzed and graphed using GraphPad Prism9. An unpaired t-test as well as an outlier's test was used to determine statistical significance. A p-value less than 0.05 was considered significant.

### Results

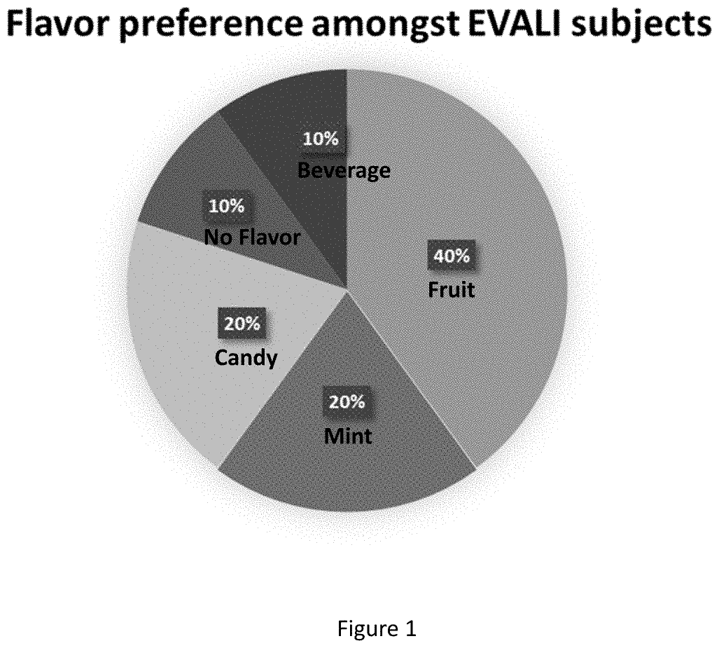

Six age- and sex-matched subjects with confirmed EVALI (n=6) and non-users (n=6) were included for biomarker testing. Baseline demographics, and if applicable, vaping status and hospitalizations details were obtained and summarized in Table 3. The average age of non-users and EVALI subjects was 22 years. The gender breakdown for both groups was 50% female to male. Non-users and EVALI subjects were mostly identified as Caucasian (Table 3). The clinical presentation and median peak laboratory values for EVALI subjects were collected and summarized in Tables 1 and 2, respectively. All EVALI participants required hospitalization and met clinical criteria for ‘confirmed’ EVALI (Kalininskiy et al., Pediatr Med 2021, 4). Primary chief complaints were GI-related (3/6), respiratory (2/6), or combined (3/6) for EVALI subjects. Maximal respiratory support included supplemental oxygen (5/6) or invasive positive pressure (1/6). One EVALI subject disclosed known drug abuse and reported hypertriglyceridemia in the past medical history. All subjects received systemic steroids (median: 9 days). Based on the self-reported questionnaire, EVALI subjects had a preference for fruit-flavored e-cigs, followed by mint and candy flavors. Beverage and no flavor were also reported (FIG. 1).

Plasma Cotinine and THC Metabolite Levels in Plasma and Urine Samples from EVALI Subjects

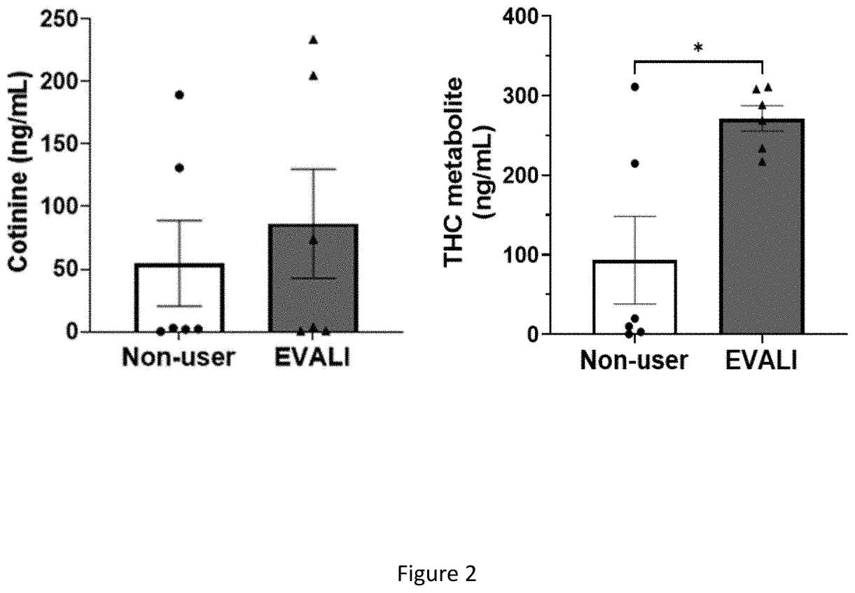

Previous studies report high incidences of THC-based cartridges and associated e-cig products among those hospitalized with EVALI (Adhikari et al., Cureus 2021, 13(2); Muthumalage et al., Toxics 2020, 8(3); Song et al., Cancer Epidemiology, Biomarkers, and prevention 2020, 29(2):443-451). Considering this, all urine and plasma samples were evaluated for THC metabolite, i.e., (−)-11-nor-9-carboxy-Δ⁹-THC and the nicotine metabolite, i.e., cotinine, respectively. Consistent with these prior reports, urinary levels of the THC metabolite were significantly elevated in EVALI subjects compared to non-users (p=0.0112, FIG. 2). Plasma cotinine levels were also elevated in EVALI subjects, but not statistically different from non-users (p=0.5815), showing relatively low amount in EVALI users, but also suggesting that EVALI subjects are either dual users of e-cigs and tobacco cigarettes or vaped nicotine-containing e-cigs.

Oxidative Stress Markers in Plasma and Urine from EVALI Subjects

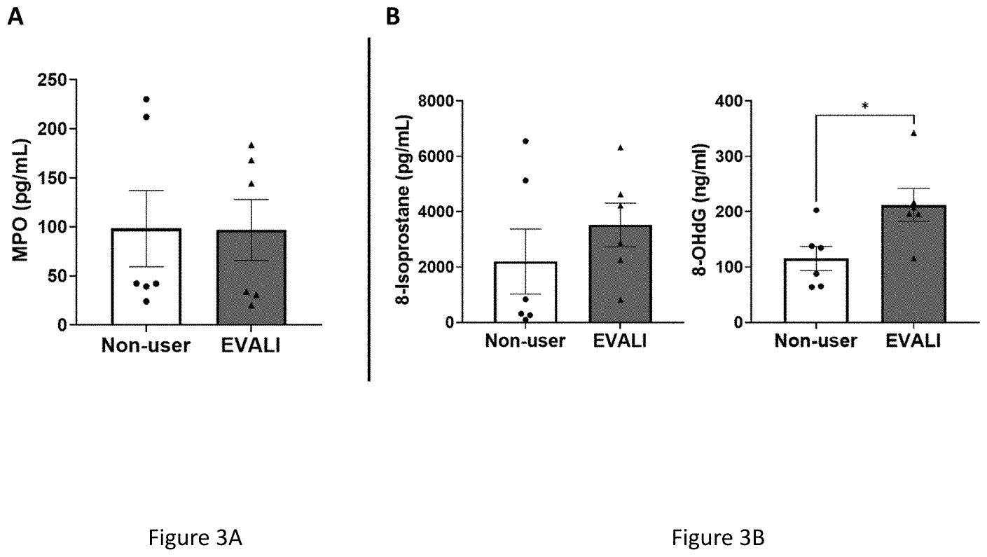

To determine if elevated reactive oxygen species (ROS)/oxidative stress secondary to vaping contributed to the hospitalization in EVALI subjects, the levels of three oxidative stress markers-myeloperoxidase (MPO), 8-isoprostane and 8-hydroxy-2r-deoxyguanosine (8-OHdG)—were quantitated in the biological samples from all subjects. MPO was not significantly different between groups (FIG. 3A). Likewise, 8-isoprostane was insignificantly elevated in the urine of EVALI subjects when compared with non-users (FIG. 3B). However, the concentration of 8-OHdG (p=0.0258) demonstrated a statistically significant increase in EVALI subjects as compared to non-users (FIG. 3B).

### Pro-Resolving Lipid Mediators in Plasma of EVALI Subjects

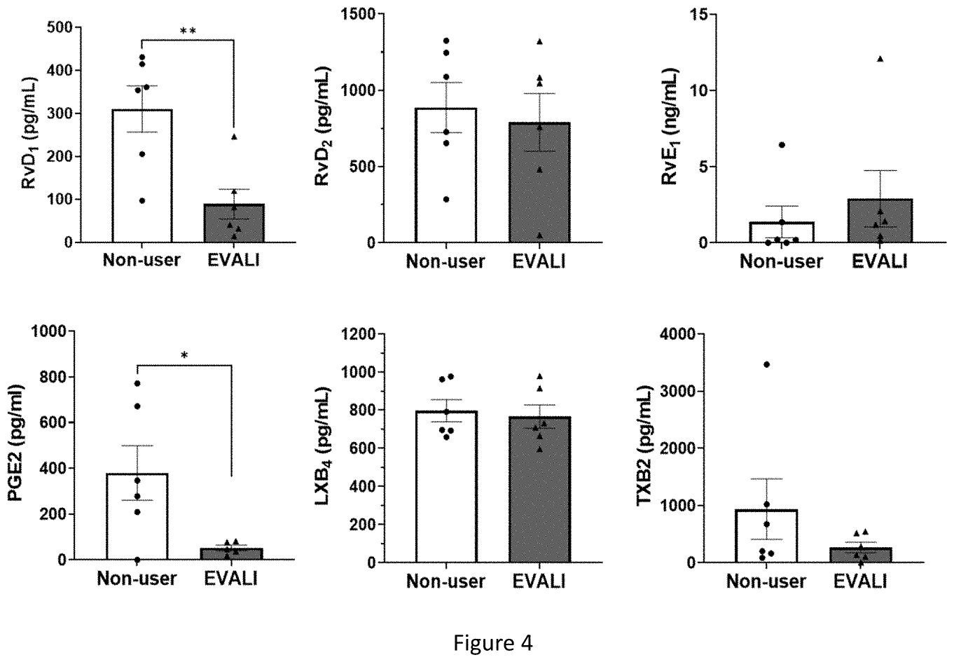

Since many mediators of inflammation are derived from phospholipids or polyunsaturated fatty acids, levels of lipid-derived mediators, including Prostaglandin E2 (PGE2), Resolvin D1 (RvD1), Resolvin D2 (RvD2), Lipoxin B4 (LXB4), Thromboxane B2 (TXB2) and Resolvin E1 (RvE1), were measured in plasma from EVALI subjects and non-user. PGE₂ (p=0.0342) levels were significantly decreased in plasma from EVALI subjects compared with non-users (FIG. 4). Similarly, RvD1 (p=0.0062) demonstrated a significantly lower concentration in EVALI subjects compared with non-users. However, other mediators, such as RvD2, RvE1, TXB2 and LXB4 exhibited non-significant changes between the groups.

### Inflammatory Biomarkers in Plasma

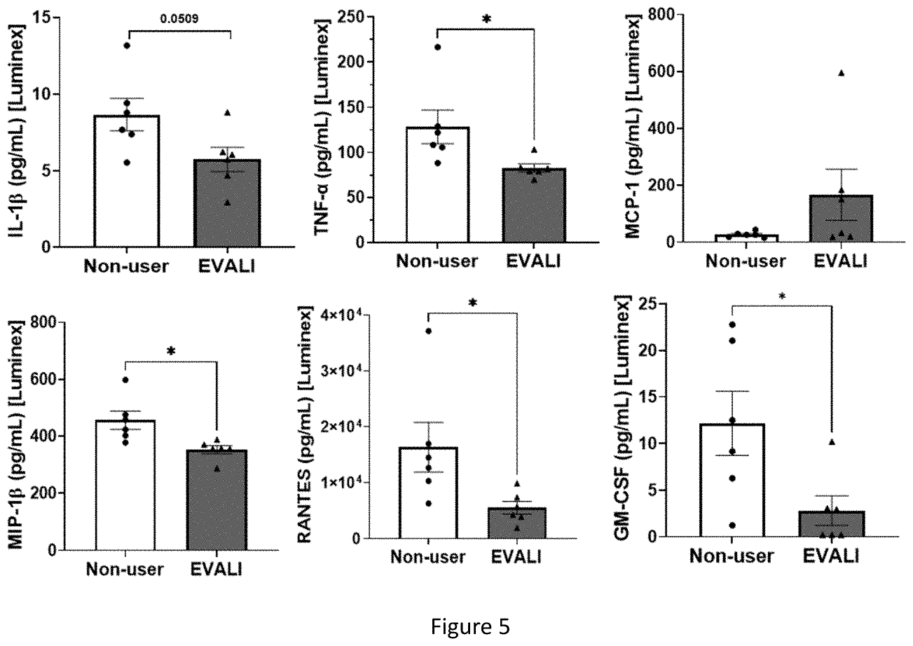

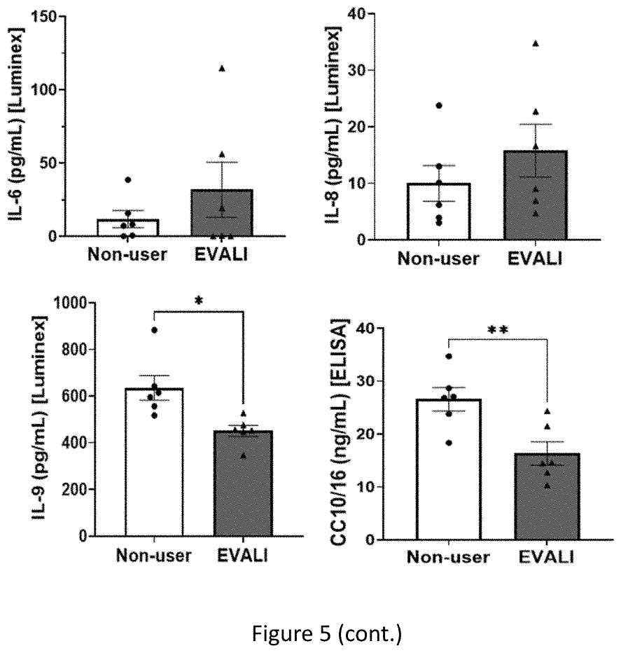

The levels of pro- and anti-inflammatory cytokines/chemokines were also measured in the plasma. Plasma levels of TNF-α, MIP-1β, RANTES, GM-CSF, IL-9 and CC10/16 demonstrated significantly lower levels in EVALI subjects than the non-users (FIG. 5). IL-1β levels, although not statistically significant, were lower in EVALI subjects compared to non-users (p=0.0509). Contrarily, we found non-significant increases between groups for MCP-1 (p=0.1528), IL-8 (p=0.3296), and IL-6 (p=0.3329). Other targets, like IL-1ra and eotaxin saw insignificant alterations in levels between EVALI subjects and non-users as well (see Table 4 below).

TABLE 4
Mean values for various biomarkers quantitated in plasma or urine from
EVALI subjects and healthy subjects.

| | Non-User (mean ± | EVALI (mean ± | |
|---|---|---|---|
| Parameters | SD); pg/mL | SD); pg/mL | p-value |
| THC | (93.1 ± 123.4) X 10³ | (271.2 ± 35.5) X 10³ | 0.0112* |
| metabolite | | | |
| (11-nor-9- | | | |
| carboxy- | | | |
| THC) | | | |
| Cotinine | (54.6 ± 76.6) X 10³ | (86.3 ± 97.9) X 10³ | 0.5815 |
| Oxidative | | | |
| Stress | | | |
| MPO | 98.3 ± 87.2 | 97.0 ± 69.7 | 0.9786 |
| 8-OHdG | (115.4 ± 49.0) X 10³ | (212.5 ± 66.9) X 10³ | 0.0258* |
| 8-Isoprostane | (2.20 ± 2.62) X 10³ | (3.52 ± 1.77) X 10³ | 0.3720 |
| Lipid | | | |
| Mediators | | | |
| RvD₁ | 310.3 ± 120.1 | 88.8 ± 78.5 | 0.0062** |
| RvD₂ | 887.1 ± 366.3 | 790.2 ± 423.6 | 0.7071 |
| RvE₁ | (1.36 ± 2.32) X 10³ | (2.90 ± 4.17) X 10³ | 0.4880 |
| LXB₄ | 796.3 ± 129.4 | 766.0 ± 136.8 | 0.7264 |
| PGE₂ | 379.3 ± 265.7 | 52.4 ± 24.2 | 0.0342* |
| TXB₂ | 937.3 ± 1179.6 | 266.8 ± 204.9 | 0.2390 |
| Inflammatory | | | |
| Mediators | | | |
| TNFα | 128.4 ± 41.5 | 83.0 ± 10.1 | 0.0389* |
| IL-1β | 8.66 ± 2.35 | 5.75 ± 1.76 | 0.0509 |
| MCP-1 | 26.7 ± 9.1 | 167.0 ± 202.6 | 0.1528 |
| IL-8 | 10.0 ± 7.1 | 15.8 ± 10.5 | 0.3296 |
| IL-6 | 11.9 ± 13.1 | 32.0 ± 42.1 | 0.3329 |
| CC10/16 | (26.6 ± 4.9) X 10³ | (16.3 ± 4.9) X 10³ | 0.0083^{*,#} |
| MIP1β | 455.4 ± 71.5 | 352.3 ± 31.3 | 0.0145* |
| RANTES | 16309.0 ± 9912.3 | 5507.1 ± 2573.93 | 0.0401* |
| GM-CSF | 12.2 ± 7.7 | 2.8 ± 3.5 | 0.0330* |
| Basic FGF | 124.0 ± 14.3 | 99.2 ± 25.5 | 0.0861 |
| VEGF | 182.9 ± 356.6 | 2.1 ± 0 | 0.2833 |
| PDGF-BB | 4726.4 ± 3330.7 | 1419.5 ± 1414.1 | 0.0682 |
| IL-1rα | 193.1 ± 367.3 | 403.5 ± 502.3 | 0.4671 |
| IL-2 | 5.39 ± 4.98 | 3.76 ± 2.35 | 0.5213 |
| IL-4 | 5.54 ± 1.47 | 4.54 ± 1.06 | 0.2473 |
| IL-5 | 24.6 ± 26.4 | 5.6 ± 6.5 | 0.1482 |
| IL-7 | 50.9 ± 18.0 | 56.8 ± 4.5 | 0.4961 |
| IL-9 | 635.2 ± 118.0 | 451.4 ± 53.8 | 0.0100* |
| IL-10 | 1.26 ± 1.91 | 1.86 ± 1.33 | 0.5731 |
| IL-13 | 3.58 ± 0.59 | 4.25 ± 0.93 | 0.2078 |
| IL-15 | 219.4 ± 289.7 | 9.0 ± 0 | 0.1354 |
| IL-17 | 22.2 ± 4.2 | 19.5 ± 5.1 | 0.3804 |
| Eotaxin | 71.3 ± 27.7 | 43.6 ± 23.0 | 0.1167 |
| IFNγ | 29.4 ± 11.3 | 24.2 ± 6.5 | 0.3979 |
| G-CSF | 163.1 ± 62.7 | 130.8 ± 22.9 | 0.3051 |
| IL-12p70 | 8.21 ± 4.61 | 6.32 ± 4.09 | 0.5087 |
| MIP-1α | 3.47 ± 1.43 | 3.97 ± 1.79 | 0.6326 |
| IP10 | 756.0 ± 267.9 | 2293.5 ± 2177.0 | 0.1481 |

THC metabolite, 8-OHdG, 8-isoprostane were measured in urine, whereas all other mediators were measured in plasma.
*p < 0.05; #p < 0.00125 (FDR—p < 0.05/40)

### Plasma Growth Factor Levels

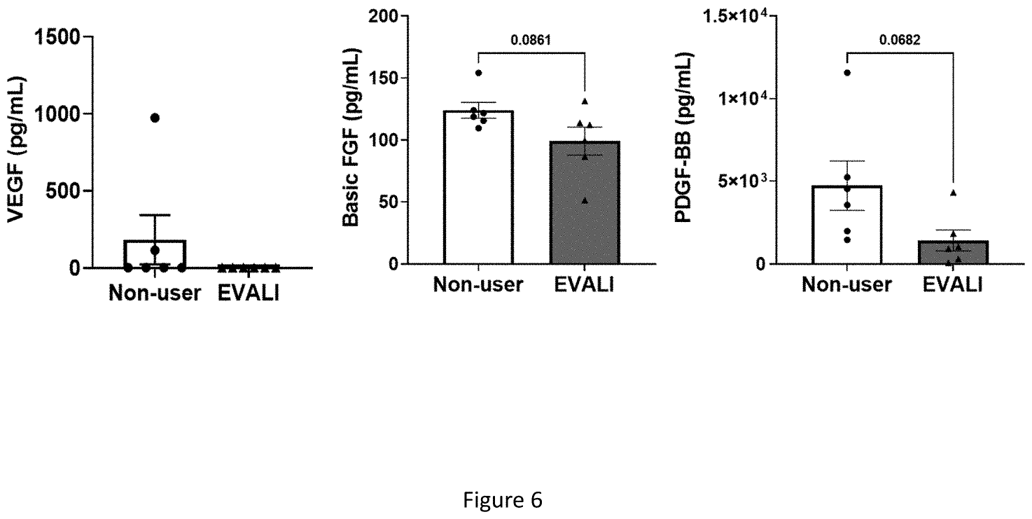

PDGF-BB and B-FGF concentrations in plasma, while not statistically significant, were lower in EVALI subjects than their non-user counterparts (FIG. 6). In contrast, VEGF levels were decreased, albeit insignificantly, for EVALI subjects when compared with non-users.

### DISCUSSION

In this study, plasma and urine from EVALI subjects and non-users were used in an effort to identify potential biomarkers of disease. Multiple inflammatory cytokines, pro-resolving lipid mediators, oxidative stress and DNA damage markers were significantly different between EVALI subjects and non-user controls.

Evidence from the literature suggest that THC-based cartridge and associated e-cig product users are more susceptible to hospitalization due to EVALI (Muthumalage et al., Toxics 2020, 8(3)). In the present self-reporting survey, only one of the subjects with EVALI admitted to using THC or CBD-based products for vaping. However, on urine analysis, multiple EVALI subjects, as well as non-user controls, demonstrated elevated levels of THC metabolite—(−)-11-nor-9-carboxy-Δ9-THC. These findings are in agreement with a previous self-reported public-survey that shows that EVALI subjects typically use THC-containing products more than e-cig users who did not develop lung injury (Navon et al., Morbidity and Mortality Weekly Report (MMWR) 2019, 68(45):1034-1039). They also found nicotine use to be more prevalent in those who did not develop EVALI (Navon et al., Morbidity and Mortality Weekly Report (MMWR) 2019, 68(45):1034-1039). However, multiple EVALI subjects in the present study had slightly higher cotinine levels as compared to non-users. In fact, previous reports have also found significantly higher levels of cotinine amongst e-cig users when compared to non-users, with levels comparable to traditional smokers (Singh et al., ERJ Open Res 2019, 5(4); Thomas et al., Cancer Epidemiol Biomarkers Prev 2020, 29(1):127-132; Park et al., Tob Induc Dis 2019, 17:12). The present results confirm that dual users of both THC- and nicotine-containing products may place oneself at greater risk for hospitalization due to EVALI.

A significant increase in the levels of 8-OHdG in urine from EVALI subjects when compared to non-users was found. Similar findings were reported in e-cig users (Singh et al., ERJ Open Res 2019, 5(4); Sakamaki-Ching et al., BMJ Open Respir Res 2020, 7(1)) and cigarette smokers (Qing et al., BMC Cancer 2019, 19(1):997). High levels of 8-OHdG indicate increased oxidative DNA damage and is a factor in the promotion of carcinogenesis (Chen et al., Environ Health Prev Med 2015, 20(5):318-324). Previous work found an increase in the levels of 8-isoprostane in the urine from e-cig users as compared to non-users (Singh et al., ERJ Open Res 2019, 5(4)). However, no significant change was observed in the levels of MPO and 8-isoprostane (though there was a trend for increase) in the biological samples from EVALI subjects and non-users. The presence of increased levels of 8-OHdG supports the notion that DNA damage plays a potentially important role in the pathogenesis of EVALI.

Resolvins D1 (RvD1) and D2 (RvD2) play a major role in dampening inflammation. Singh et al. previously demonstrated a significant decrease in the levels of RvD1 in the plasma from e-cig users (Singh et al., ERJ Open Res 2019, 5(4)). The levels of plasma RvD1 were significantly decreased in EVALI subjects compared to non-user controls. RvD1 is a lipid mediator that functions to dampen polymorphonuclear leukocytes infiltration and transmigration. Thus, lower levels of RvD1 are in line with prominent serum neutrophilia seen in the majority of EVALI subjects hospitalized with respiratory insufficiency, and may potentially contribute to failed lung repair after e-cig exposure (Singh et al., ERJ Open Res 2019, 5(4)). In contrast, a significant decrease in PGE2 levels was also found in EVALI subjects compared to controls. PGE2 induces inflammation through mast cell activation. One potential explanation for these dichotomous findings is the concurrent use of non-steroid anti-inflammatory drugs (NSAIDs) in those hospitalized with EVALI, which would falsely lower the PGE2 levels seen in this group.

Cigarette smoke-induced oxidative stress is shown to activate the inflammatory response by upregulating cytokines, such as IL-6 and IL-8. IL-6 plays a key role in mediating acute phase response and is a prognostic biomarker in various acute organ injuries, including the lung. IL-8 functions in the chemotaxis and eventual phagocytosis of neutrophils and other granulocytes. Neutrophils are linked to inflammatory lung diseases including COPD, asthma, bronchiolitis, respiratory distress syndrome and interstitial pneumonia (Fujimori et al., Respirology 2003, 8(1):33-40). With multiple assays, IL-6 and IL-8 levels were found to be comparable between EVALI and non-users. When BEAS-2B cells were exposed to counterfeit cartridges, there were significantly higher levels of IL-6 and IL-8 as compared to cells exposed to air, thus suggesting the eliciting of immune response of e-cig exposure (Muthumalage et al., Toxics 2020, 8(3)). Similarly, Singh et al. also reported a significant increase in IL-6 and IL-8 production among e-cig users when comparing the plasma inflammatory profiles with non-users (Singh et al., ERJ Open Res 2019, 5(4)). Multiple explanations may account for these differences. One explanation is that immune suppression occurs through different cytokine pathways than that of e-cig exposure alone. A second possibility is that hospitalized EVALI subjects often presented later in the course of the disease and after their treatments, peak IL-6 and IL-8 levels may have dissipated at the time of blood collection.

IL-9 is an activator of mast cells (Noelle et al., Nat Rev Immunol 2010, 10(10):683-687) and is significantly lowered in EVALI subjects compared to non-users. This interleukin has been linked to allergic asthma in mice (Temann et al., The Journal of Experimental Medicine 1998, 188(7):1307-1320), cancer (Gerlach et al., J Mol Med (Berl) 2019, 97(10):1377-1383), human allergic lung inflammation and contribute to autoimmune disease (Noelle et al., Nat Rev Immunol 2010, 10(10):683-687). IL-9 also has the ability to effect IL-13 and therefore affect epithelial cells of the lung and gut (Noelle et al., Nat Rev Immunol 2010, 10(10):683-687).

IL-1β, a pro-inflammatory cytokine, is linked to many acute and chronic inflammatory diseases, including acute lung injury. In this study, plasma IL-1β levels were lower, though not significant, for EVALI subjects when compared to the non-users. The present results are contrary to previous literature where IL-1β levels were significantly higher in BALF and saliva from e-cig users as compared to never smokers (Song et al., Cancer Epidemiology, Biomarkers, and prevention 2020, 29(2):443-451; Singh et al., ERJ Open Res 2019, 5(4)). However, when analyzed in plasma samples, there was a non-significant decrease in e-cig users (Singh et al., ERJ Open Res 2019, 5(4)). This proves that the measurement of various pro-inflammatory mediators might vary based on the biological sample being tested as well as the time of collection.

MIP-1β, also known as CCL4, is a chemokine known to play a critical role in the chemotactic activity of monocytes through the CCR5 receptor, which has been connected to diverse immune responses. MIP-1β levels were found to be increased in natural killer cells, CD8+ T cells and CD4+ T cells in pregnant women exposed to the influenza A virus. Additionally, their levels correlate with the severity of influenza symptoms and viral replication along with a similar rise in kinetics after influenza infection like other chemokines. As a whole, the MIP family is implicated as important mediators of lung disease. RANTES is a chemokine involved in leukocyte influx and bronchial hyper-responsiveness (Conti et al., Allergy Asthma Proc 2001, 22(3):133-137). It has been established to play an important role in lung allergic inflammation and leukocyte infiltration (Conti et al., Allergy Asthma Proc 2001, 22(3):133-137). In the present study, MIP-1β and RANTES were significantly lowered in EVALI subjects compared to non-users.

Club cell secretory protein (CCSP or CC10/16) has several immune-regulatory activities, including inhibition of phospholipase A2 (Kropski et al., Chest 2009, 135(6):1440-1447; Barnes et al., CHEST 2015, 147(6):1447-1448). In past studies of COPD, asthma, idiopathic pulmonary fibrosis, sarcoidosis and other pulmonary issues, CC16 has shown promise as a potential biomarker of lung epithelial injury (Kropski et al., Chest 2009, 135(6):1440-1447; Almuntashiri et al., J Clin Med 2020, 9(12)). Kropski et al. found lower levels of CC16 in the plasma of acute lung injury patients when compared to acute cardiogenic pulmonary edema patients (Kropski et al., Chest 2009, 135(6):1440-1447). Not unexpectedly, when previously analyzed in e-cig users, there was only a minimal difference compared to normal subjects in both plasma and urine (Singh et al., ERJ Open Res 2019, 5(4)). In the present study, it was found that EVALI subjects have significantly lower CC16 levels than their non-user counterparts. This is most likely due to the difference in severity at the time of presentation, with most EVALI subjects requiring hospitalization. It is possible that the lower levels of CC16 are secondary to alterations in alveolar epithelial permeability, club cell death, or changes in transcriptional activity within the remaining club cells (Kropski et al., Chest 2009, 135(6):1440-1447). However, CC16 remains a significant and biologically relevant biomarker for future investigation.

A potent inflammatory cytokine, TNF-α, has been implicated in various pulmonary diseases like asthma, and COPD/emphysema. In a previous study, smaller concentrations of TNF-α in e-cig users compared to non-users, albeit non-significant, was found (Singh et al., ERJ Open Res 2019, 5(4)). This has concurred in the present study, where this was found to be true of EVALI subjects with the same level of non-significance.

MCP-1, which is also known as CCL2, is a chemokine that regulates the migration and infiltration of monocytes and macrophages in innate immunity (Deshmane et al., Journal of interferon and Cytokine Research 2009, 29(6):313-326). This chemokine has been linked to atherosclerosis, inflammatory bowel disease, asthma and arthritis (Deshmane et al., Journal of interferon and Cytokine Research 2009, 29(6):313-326). It was also connected to interstitial lung disease in pediatric patients with high levels, negatively correlating to restrictive lung function, forced vital capacity, total lung capacity and other lung disease severity scores (Hartl et al., Respir Res 2005, 6:93). In a previous study, the levels of MCP-1 in non-users and e-cigs users were indistinguishable from each other (Singh et al., ERJ Open Res 2019, 5(4)). Non-significant higher concentrations were observed in EVALI subjects compared to non-users in Luminex-based assays.

Platelet-derived growth factor (PDGF) mediates airway inflammation, remodeling in asthma, and plays a significant role in blood vessel formation (Kardas et al., Front Pharmacol 2020, 11:47). Cucina et al. established that nicotine enhanced the release of PDGF-BB in endothelial cells (Cucina et al., J Surg Res 2000, 92(2):233-238). Concurring with this, Singh et al. found the AA isoform to be significantly elevated in e-cig users, which established high levels of cotinine as well (Singh et al., ERJ Open Res 2019, 5(4)). In contrast, the present study found that the PDGF-BB isoform was insignificantly reduced in EVALI subjects as compared to controls when an increase in nicotine amongst EVALI subjects was also shown.

Overall, the present study identified multiple potential EVALI biomarkers, including markers of oxidative stress such as 8-OHdG and 8-isoprostane as well as reduced levels of pro-resolving lipid mediator, RvD1 and anti-inflammatory airway epithelial marker, CC10/16. These findings provide a strong basis for the use of these biomarkers in the diagnosis of EVALI.

### Example 2: Phosphatidylethanolamines as Plasma Biomarkers for Diagnosing EVALI

In June 2019, a new pandemic spread throughout the United States with thousands of adolescents and young adults hospitalized with lung injury following e-cigarette use. This new clinical entity of e-cigarette, or vaping, product use-associated lung injury (EVALI) has affected over 2,800 individuals as of February 2020 (last national report by the Center for Disease Control (CDC)). In January 2020, labs affiliated with the New York State Department of Health identified vitamin E acetate (VEA) as a primary compound found in many e-cigarette/vaping cartridges from those hospitalized with EVALI (Duffy et al., Toxics. 2020; 8(1)). VEA was subsequently identified also in the bronchoalveolar lavage fluid (BALF) of those patients hospitalized with EVALI by a CDC working group (Blount et al., N Engl J Med. 2020; 382(8):697-705). Hence, VEA remains the lead chemical compound associated with EVALI.

However, a significant majority of individuals affected by EVALI do not require ICU level care (Kalininskiy et al., Lancet Respir Med. 2019; 7(12):1017-26; Hartnett et al., N Engl J Med. 2020; 382(8):766-72), and only a small minority of individuals hospitalized undergo flexible bronchoscopy with lavage (BAL), demonstrated by only 51 BAL samples from >2800 individuals affected at the pandemic's peak (Blount et al., N Engl J Med. 2020; 382(8):697-705). Only a handful of labs nationwide have the training to complete the tedious process of isolating and identify VEA from BAL. Collectively, these factors significantly limit the use of BALF VEA levels as a diagnostic biomarker of EVALI, and for most hospitalized patients, EVALI remain a diagnosis of exclusion. Here, the present study highlights the identification of plasma phosphatidylethanolamines as novel and non-invasive plasma biomarkers in diagnosing EVALI.

To identify potential biomarkers for diagnosing EVALI, three groups of subject were enrolled: non-smoking controls (n=5), e-cig users not affected by EVALI (n=5), and e-cig users hospitalized for EVALI (n=5). Written informed consent was obtained from all subjects. Age of eligibility was between 18 and 35 years. Groups were matched for age at enrollment. Exclusion criteria included: (1) prior history of heart or lung disease, diabetes, cancer, and/or current viral respiratory tract infection identified by viral PCR or blood culture (including PCR negativity for COVID-19); (2) current medical use of daily anti-inflammatories or corticosteroids; and (3) currently pregnant or breast feeding. Subjects in the non-user group were required to have never smoked or used e-cigarette or tobacco products. Subjects in the e-cig user group were not hospitalized and recruited from the community by flyer advertisement. Subjects in the EVALI group were ‘confirmed’ diagnoses (Kalininskiy et al., Lancet Respir Med. 2019; 7(12):1017-26; Jatlaoui et al., Morb Mortal Wkly Rep. 2019; 68(46):1081-6), hospitalized at time of enrollment and enrolled after February 2020 (the peak of EVALI reporting). All EVALI subject samples were also obtained prior to corticosteroid treatment.

Clinical demographics, presentation, and management are listed in Table 5 shown below. Untargeted lipidomics was performed on human plasma via lipid extraction and subsequent LC-MS/MS identification. Lipidomics was chosen considering many e-cigarette liquids, in addition to VEA, are highly viscous and lipophilic chemicals. Additionally, early data from chronic e-cig users (Wang et al., Metabolites. 2021; 11(6); Muthumalage et al., bioRxiv. 2020 Jun. 15; 2020.06.14.151381; Middlekauff et al., J Transl Med. 2020; 18(1):379) as well as from sub-chronic e-cig exposures in mice (Madison et al., J Clin Invest. 2019; 129(10):4290-304; Moshensky et al., ERJ Open Res. 2021; 7(3)) support abnormal lipid metabolism in exposed plasma samples.

TABLE 5
Demographics, clinical presentation and management of non-users
(n = 5), e-cig users (n = 5), and EVALI users (n = 5).

| | Non-users | E-cig users | EVALI users |
|---|---|---|---|
| | (n = 5) | (n = 5) | (n = 5) |
| Age, years | | | |
| Mean (SD) | 22 (2.5) | 21.5 (3.4) | 24 (6.3) |
| Range | 18-25 | 18-28 | 19-35 |
| Sex, n (%) | | | |
| Male | 3 (60) | 3 (60) | 3 (60) |
| Female | 2 (40) | 2 (40) | 2 (40) |
| E-cig use, n (%) | | | |
| Vaping Frequency | N/A | | |
| 10+ times/day | | 3 (60) | 3 (60) |
| 3-5 times/day | | 2 (40) | 2 (40) |
| <1 time/day | | 0 | 0 |
| Duration per Session | N/A | | |
| 20+ min | | 1 (20) | 0 |
| 10-14 min | | 1 (20) | 2 (40) |
| 5-9 min | | 2 (40) | 0 |
| <5 min | | 1 (20) | 3 (80) |
| Years Smoking | N/A | | |
| >5+ yr. | | 1 (20) | 0 |
| 2-5 yr. | | 1 (20) | 2 (33.3) |
| 1-2 yr. | | 3 (60) | 2 (33.3) |
| <1 yr. | | 0 (0) | 2 (33.3) |
| Hospitalizations, n (%) | 0 | 0 | 6 (100) |
| Hospital Duration | N/A | N/A | 5.5 (4-6.75) |
| (median days, IQR) | | | |
| Chief Complaint | N/A | N/A | |
| Gastrointestinal | | | 3 (60) |
| Respiratory | | | 1 (20) |
| Combination | | | 1 (20) |
| Respiratory Support | N/A | N/A | |
| Ventilation | | | 0 (0) |
| Supplemental Oxygen | | | 4 (80) |
| No Support | | | 1 (20) |
| Treatment | | | |
| Antibiotics | | | 5 (100) |
| Steroids | | | 5 (100) |

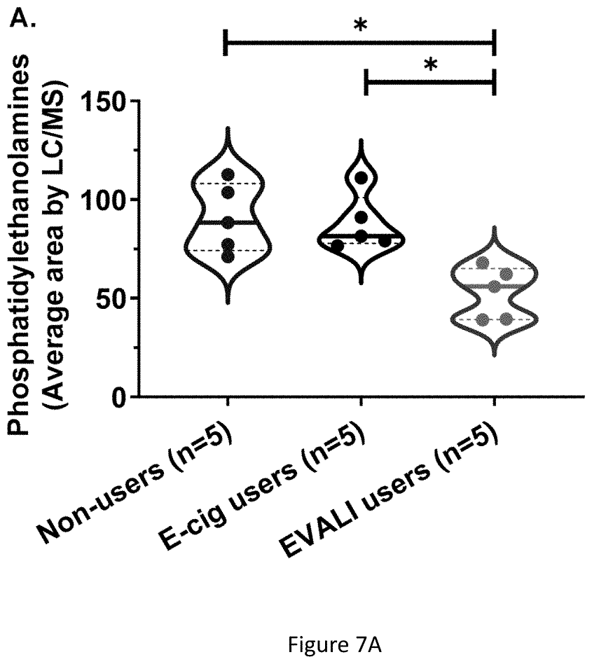

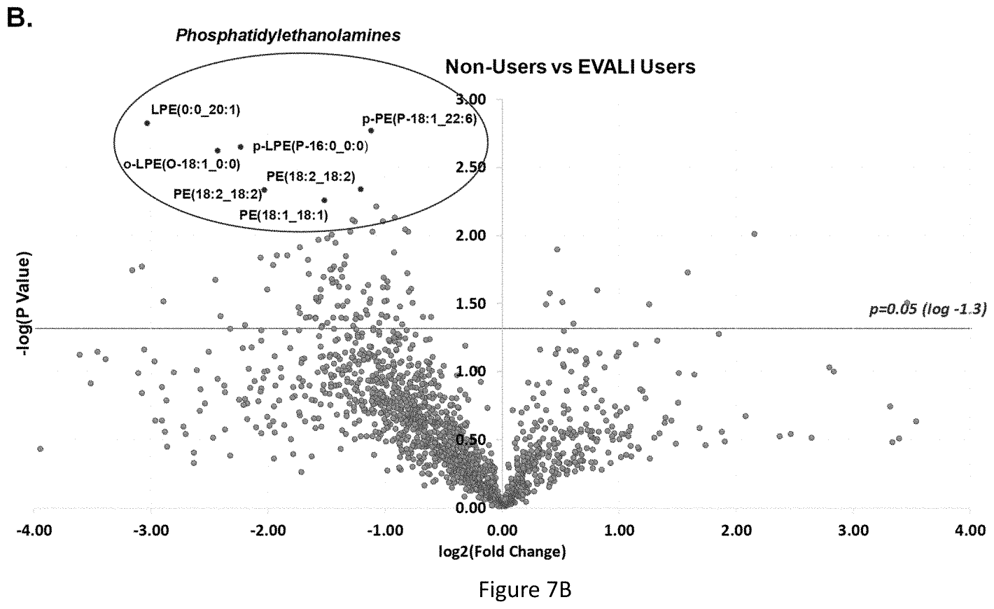

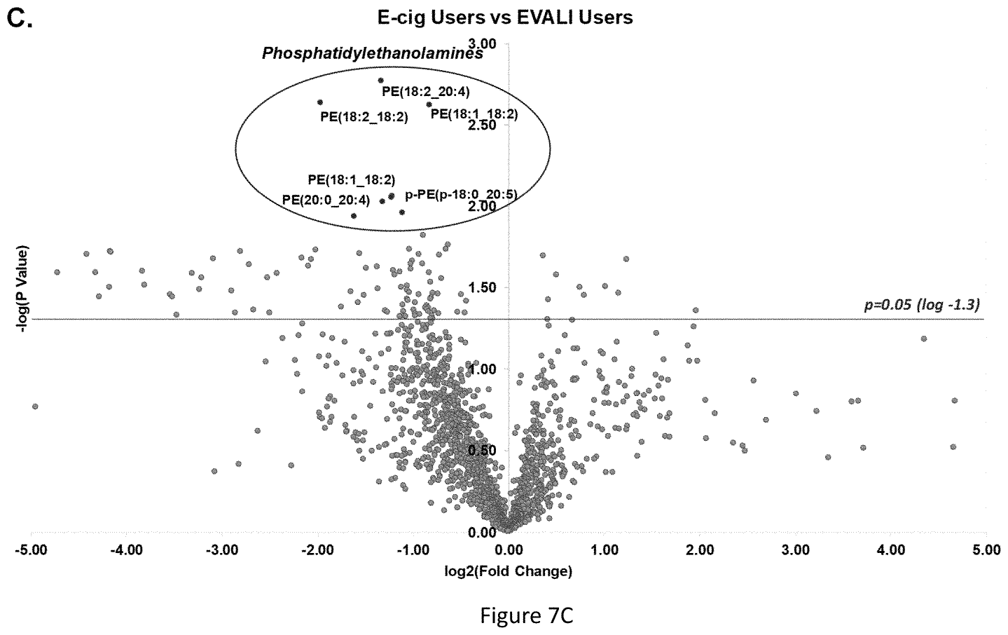

A total of 513 unique molecular species were identified from untargeted lipidomics grouped by lipid class. Principal component analysis of all lipid classes failed to delineate separation between groups. However, in lipid subclass analysis, phosphatidylethanolamines (PEs) were significantly reduced in all EVALI subjects compared to control groups of non-users and e-cig users (FIG. 7A, Kruskal-Wallis, **p=0.0029). The median plasma level of PE's in EVALI users was 56.0 (IQR: 39.3-65.2) while 88.4 (IQR: 74.3-108.2) and 81.5 (IQR: 77.9-101.1) in non-users and e-cig users, respectively. In other words, median plasma levels of PE were 36.7% and 31.3% reduced in EVALI versus non-users and EVALI versus e-cig users, respectively. Specific plasma PEs reduced in EVALI subjects compared to non-users and e-cig users are highlighted in volcano plot comparisons between individual groups (FIGS. 7B and 7C). Other structurally similar phospholipids (PLs), such as certain phosphatidylcholines, phosphatidylserines and phosphoinositols, were also identified as reduced when comparing individual lipid components between groups, however individual sub-class analysis did not differ significantly between groups.

In a group of hospitalized EVALI subjects and prior to corticosteroid treatment, plasma phosphatidylethanolamines (PEs) levels were reduced by greater than 30% compared to PE's levels of non-users and e-cig users. PEs are a class of phospholipids found in all human cells composing ˜20% of all PL's within the cell membrane (Vance et al., Biochim Biophys Acta. 2013; 1831(3):543-54; Wellner et al., Biochim Biophys Acta. 2013; 1831(3):652-62). In conjunction with phosphatidylcholine, PEs are classified as plasmalogens based on their lipid structure containing a vinyl ether bond at the sn-1 position and an ester bond at sn-2 position of the glycerol backbone (Nagan et al., Prog Lipid Res. 2001; 40(3):199-229). Plasmalogens are most abundant in the nervous, immune, and cardiovascular systems, but are also commonly seen in the kidneys, skeletal muscles, and lungs (Braverman et al., Biochim Biophys Acta. 2012; 1822(9):1442-52). Three of the most common biologic functions of plasmalogens are cell membrane stability, oxidative potential, and reservoirs for second messengers (Braverman et al., Biochim Biophys Acta. 2012; 1822(9):1442-52).

Specific to the lung, plasmalogens play an essential role in surfactant homeostasis. Plasmalogens help to reduce surface tension and viscosity of surfactant in combination with cholesterol (Lohner et al., Biochim Biophys Acta. 1991; 1061(2):132-40; Rudiger et al., Am J Physiol. 1998; 274(1):L143-8). Lohner et al (1991) first demonstrated that plasmalogens promote and stabilize the non-lamellar membrane phase structure of surfactant. Rudiger et al (1998) showed the addition of plasmalogens to surfactant-like PL mixtures resulted in lower surface tensions, emphasizing the role of plasmalogens in surfactant's function of preventing alveolar collapse. Additionally, in preterm infants who received surfactant supplemented with higher plasmalogen content, the likelihood of developing bronchopulmonary dysplasia was reduced (Rudiger et al., Crit Care Med. 2000; 28(5):1572-7). Related to oxidative potential, PE's are preferentially oxidized over other diacyl phospholipids functioning also as an antioxidant when in the presence of free radicals and singlet oxygen (Broniec et al, Free Radic Biol Med. 2011; 50(7):892-8). When mouse surfactant was exposed to ozone, plasmalogen content was reduced ˜50% prior to any significant change in other diacyl PL concentration, further supporting that plasmalogens function both in surface tension reduction as well as an antioxidant for the lung.

Plasmalogens may have a role in the pathogenesis of EVALI. Related to EVALI, Dipasquale et al. (2020) recently showed VEA significantly changed the mechanical properties of two surfactant mimics in vitro (DiPasquale et al., Chem Res Toxicol. 2020; 33(9):2432-40). Both of these surfactant mimics contained plasmalogens. Using neutron spin echo spectroscopy, increasing concentrations of VEA nonlinearly increased membrane fluidity and area compressibility. Modulation of these two properties in the presence of VEA increased surface tension, resulting in monolayer collapse. The reduced concentrations of plasmalogens identified herein from the plasma of EVALI subjects may be physiologically relevant to the respiratory insufficiency seen in those subjects hospitalized with hypoxemia and bilateral ground glass opacities. Specifically, reduced plasmalogen concentration of lung surfactant would predispose EVALI subjects to alveolar collapse from increased surface tension of surfactant. However, surfactant composition from subjects with EVALI would help strengthen this association between reduced circulating plasmalogen content and respiratory insufficiency due to EVALI cartridge exposures.

Additional studies are underway to validate PEs as non-invasive biomarkers for diagnosing EVALI. Defining non-invasive biomarkers in the diagnosis of EVALI is essential considering flexible bronchoscopy remains elective in the routine evaluation and management of EVALI and presentation is nearly identical to other forms of acute lung injury. Unique to this study, all EVALI subjects were enrolled after CDC stopped reporting. Similar to others (Kligerman et al., Chest. 2021; 160(4):1492-511), institutions continue to see multiple patients hospitalized for EVALI after February 2020, emphasizing the continued importance and need for non-invasive biomarkers for this new disease that remains a diagnosis of exclusion and significantly different from other forms of acute lung injury.

### Example 3: Biomarkers for Identifying E-Cigarette, or Vaping, Product Use-Associated Lung Injury (EVALI): Urine Metabolites

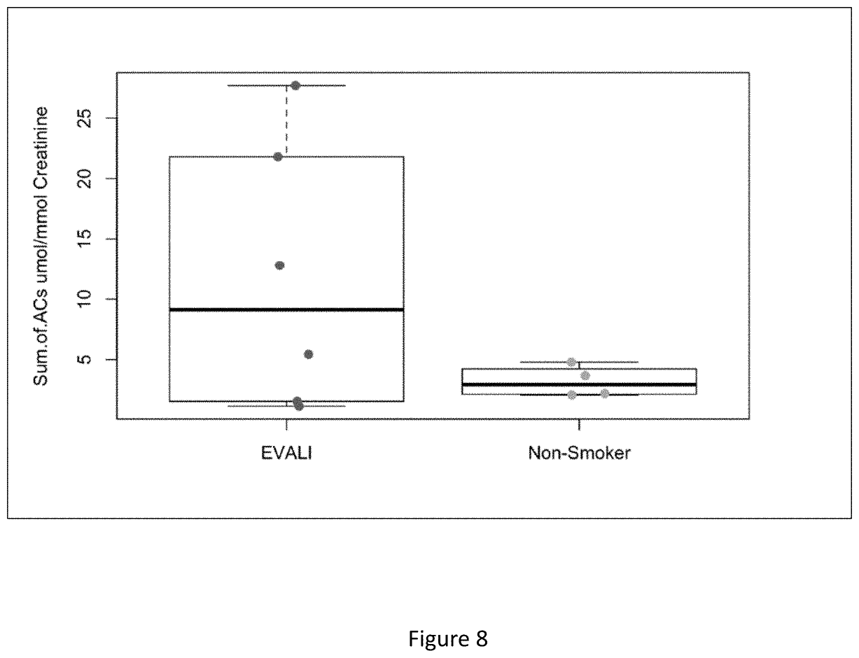

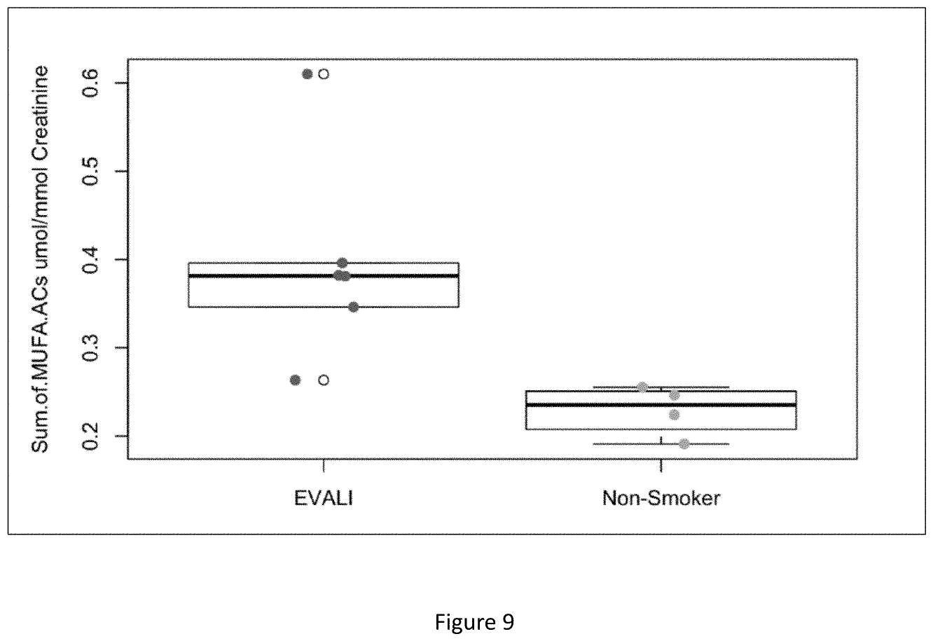

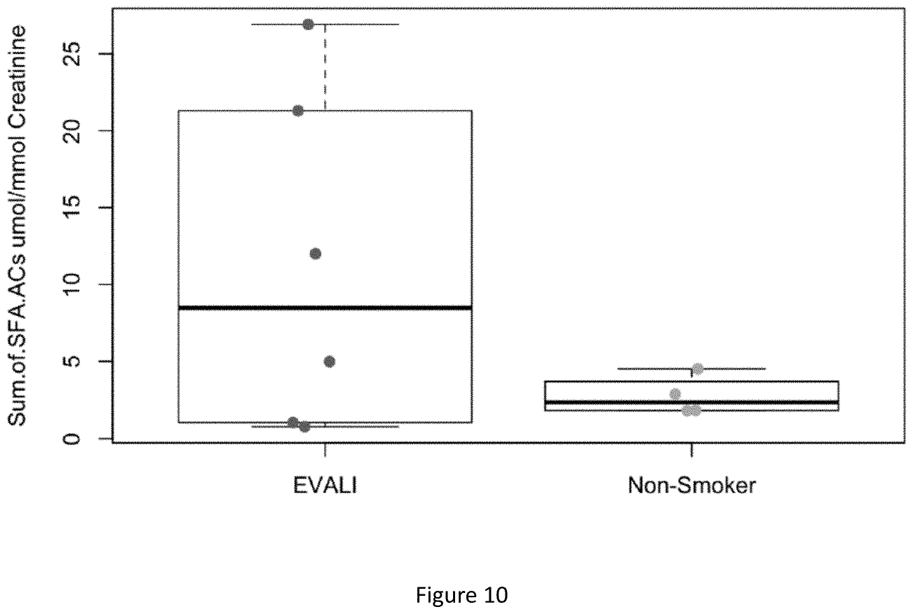

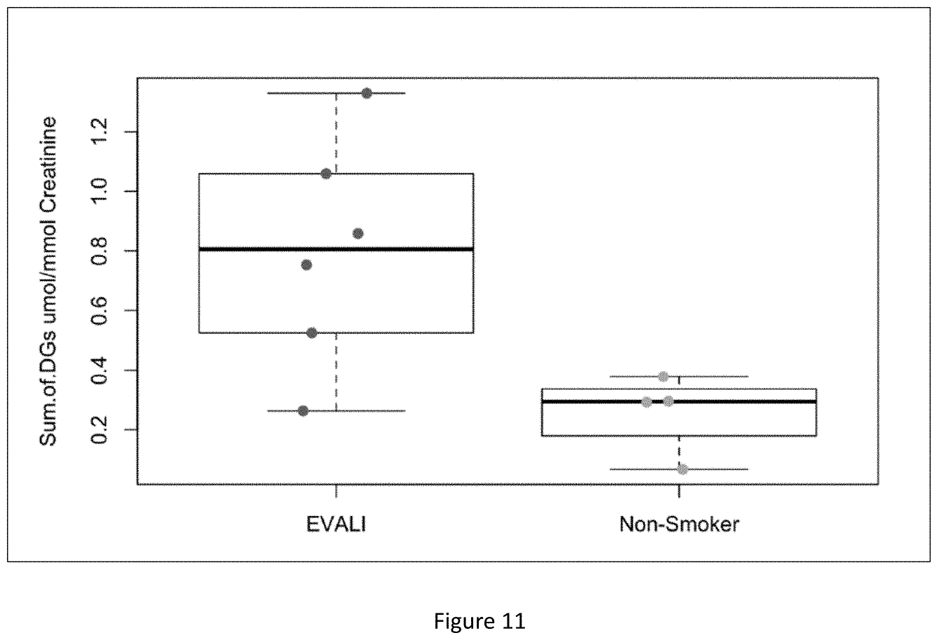

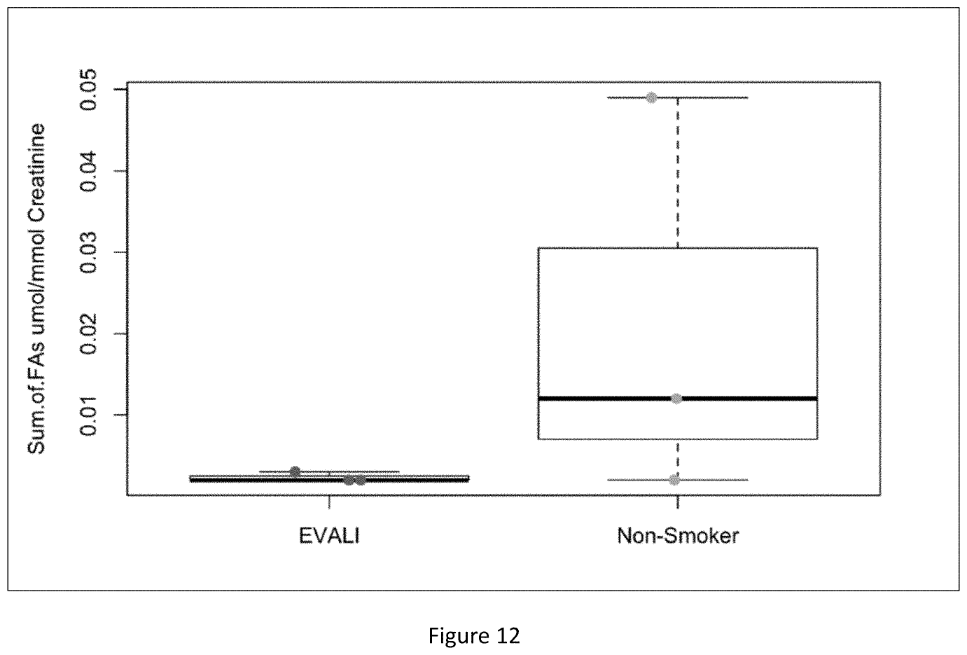

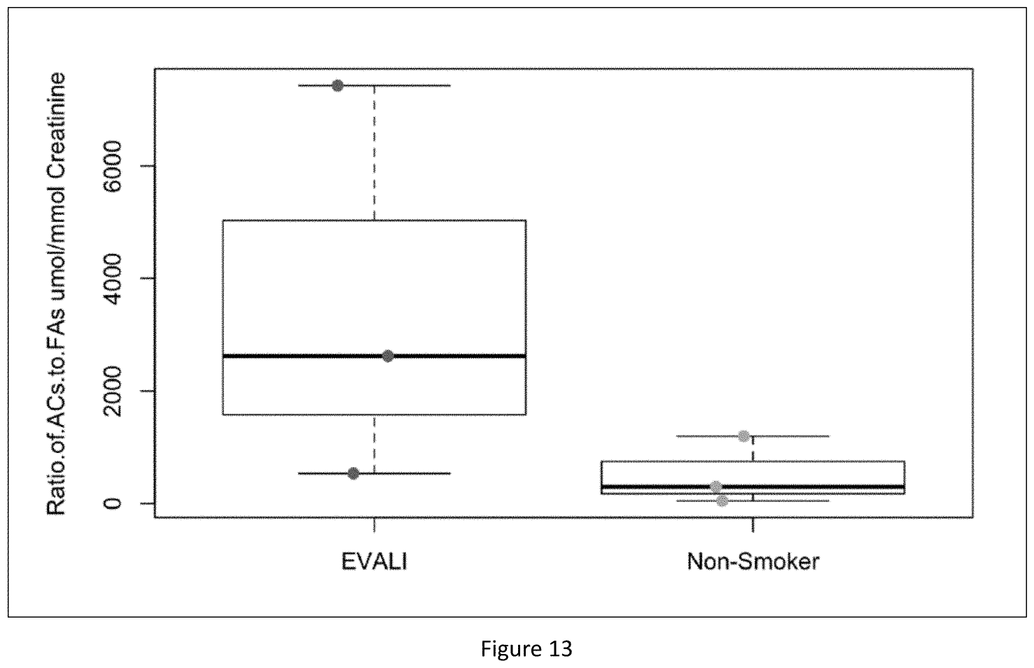

Over 100 urine metabolites were tested on EVALI subjects and controls using biocrates MxP® Quant 500 technology. Metabolites that showed a difference between EVALI and control subjects were selected for further study. All urinary levels reported in μmol/mmol Creatinine (Cr). The urinary metabolite biomarkers that were included for further evaluation included: sum of acylcarnitine (AC) (FIG. 8), sum of monounsaturated fatty acid (MUFA) (FIG. 9), sum of saturated fatty acid (SFA) (FIG. 10), sum of diglycerides (DG) (FIG. 11), sum of fatty acid (FA) (FIG. 12) and the ratio of AC(sum):FA(sum) (FIG. 13). It is noteworthy that there are reduced FA but elevated ratio AC(sum):FA(sum) in EVALI subject vs. controls.

The data demonstrate that there is an elevation in urinary AC(sum), MUFA(sum), SFA(sum), and DG(sum) in EVALI vs. controls, reduced urinary FA(sum) in EVALI vs. controls, and an elevation in urinary AC(sum):FA(sum) ratio in EVALI vs. controls. Overall the data demonstrate that fatty acid beta oxidation products can be used as biomarkers for EVALI.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying e-cigarette, or vaping, product use associated lung injury (EVALI) in a subject, the method comprising:
a) obtaining a biological sample of said subject;
b) measuring the level of one or more EVALI-associated biomarkers in said biological sample;
c) comparing the level of said one or more EVALI-associated biomarkers in said biological sample to the level of one or more EVALI-associated biomarkers in a comparator; and
d) identifying said subject as having EVALI when the level of one or more EVALI-associated biomarker in said biological sample of the subject substantially differs from the level of one or more EVALI-associated biomarker in said comparator.

2. The method of claim 1, wherein said biological sample comprises one or more selected from the group consisting of: whole blood, serum, plasma, urine, and saliva.

3. The method of claim 1, wherein said measuring comprises the use of one or more technique selected from the group consisting of: liquid chromatography with tandem mass spectrometry (LC-MS/MS), enzyme-linked immunosorbent assay (ELISA), bead-based multiplex immunoassay, and polymerase chain reaction.

4. The method of claim 1, wherein said EVALI-associated biomarker comprises one or more molecule selected from the group consisting of: 11-nor-9-carboxy-tetrahydrocannabinol, 8-hydroxy-2′-deoxyguanosine, Prostaglandin E₂, and Resolvin D₁.

5. The method of claim 1, wherein said EVALI-associated biomarker comprises one or more protein selected from the group consisting of: tumor necrosis factor alpha (TNFα), macrophage inflammatory protein beta (MIP1β), Regulated upon Activation, Normal T Cell Expressed and Presumably Secreted (RANTES), granulocyte-macrophage colony-stimulating factor (GM-CSF), interleukin 9 (IL-9), and club cell protein 10/16 (CC10/16).

6. The method of claim 1, wherein said EVALI-associated biomarker comprises a nucleic acid molecule encoding one or more selected from the group consisting: TNFα, MIP1β, RANTES, GM-CSF, IL-9, and CC10/16.

7. The method of claim 1, wherein said EVALI-associated biomarker comprises a lipid.

8. The method of claim 7, wherein the lipid comprises one or more phospholipid selected from the group consisting of: a phosphatidylethanolamine (PE), a phosphatidylcholine, a phosphatidylserine and a phosphoinositol.

9. The method of claim 7, wherein said PE comprises one or more selected from the group consisting of: 1-linoleoyl-2-arachidonoyl-sn-glycero-3-phosphoethanolamine [PE(18:2/20:4)], 1,2-dilinoleoyl-sn-glycero-3-phosphatidylethanolamine [PE(18:2/18:2)], 1-oleoyl-2-linoleyl-sn-glycero-3-phosphoethanolamine [PE(18:1/18:2)], 1-arachidonyl-2-arachidonoyl-sn-glycero-3-phosphoethanolamine [PE(20:0/20:4)], 1-stearoyl-2-eicosapentaenoyl-sn-glycero-3-phosphoethanolamine [PE(18:0/20:5)], PE(P-18:0/20:4), PE(18:0/20:4), PE(18:0/18:2), PE(0:0/18:2), PE(0:0/20:4), PE(16:0/20:4), PE(16:0/20:4), PE(16:0/18:2), PE(16:0/22:6), PE(P-18:0/22:6), PE(0:0/18:1), PE(0:0/18:0), PE(P-16:0/22:6), PE(0:0/16:0), PE(P-16:0/18:2), PE(O-16:0/20:4), PE(18:0/18:1), PE(O-18:0/22:6), PE(18:1/20:4), PE(P-16:0/22:4), PE(0:0/22:6), p-PE(P-18:0/20:5), PE(18:0/22:6), PE(16:0/18:1), PE(P-18:1/22:6), PE(P-18:0/22:4), PE(P-20:0/20:4), PE(P-18:0/18:1), PE(P-16:0/18:1), PE(18:1/18:1), PE(18:0/20:3), p-PE(P-16:0/0:0), PE(O-18:1/0:0), PE(P-18:0/20:3), PE(P-18:0/20:4), PE(P-16:0/20:3), PE(P-16:0/22:6), PE(P-16:0/18:2), PE(P-20:0/18:2), PE(P-20:0/20:5), PE(18:1/22:6), PE(16:0/18:3), PE(0:0/20:3), PE(20:5/18:0), PE(18:0/22:4), PE(P-20:0/20:5), PE(0:0/22:5), PE(P-16:0/20:5), PE(O-16:0/18:2), PE(16:0/22:4), PE(P-18:0/22:6), PE(0:0/18:3), PE(0:0/20:5), PE(16:0/20:5), PE(0:0/16:1), PE(0:0/20:1), and PE(P-20:0/22:4).

10. The method of claim 1, wherein the sample is a urine sample, and further wherein said EVALI-associated biomarker comprises at least one selected from the group consisting of acylcarnitine (AC), monounsaturated fatty acid (MUFA), saturated fatty acid (SFA), diglycerides (DG) or fatty acid (FA).

11. The method of claim 10, wherein the biomarker is selected from the group consisting of the sum of acylcarnitine (AC), the sum of monounsaturated fatty acid (MUFA), the sum of saturated fatty acid (SFA), the sum of diglycerides (DG), the sum of fatty acid (FA), and the ratio of AC:FA.

12. The method of claim 11, wherein the biomarker is selected from the group consisting of: the sum of AC is elevated as compared to the average or normalized sum of AC in a non-smoking population, the sum of MUFA is elevated as compared to the average or normalized sum of MUFA in a non-smoking population, the sum of SFA is elevated as compared to the average or normalized sum of SFA in a non-smoking population, the sum of DG is elevated as compared to the average or normalized sum of DG in a non-smoking population, the sum of FA is decreased as compared to the average or normalized sum of FA in a non-smoking population, and the ratio of the sum of AC:the sum of FA is elevated as compared to the average or normalized ratio of the sum of AC:the sum of FA in a non-smoking population.

13. The method of claim 1, wherein the substantial difference comprises a decrease in the level of one or more EVALI-associated biomarker in the subject biological sample as compared to the level of one or more EVALI-associated biomarker in the comparator.

14. The method of claim 13, wherein the substantial difference comprises a statistically significant decrease in the level of one or more EVALI-associated biomarker in the subject biological sample as compared to the level of one or more EVALI-associated biomarker in the comparator.

15. The method of claim 1, wherein the substantial difference comprises an increase in the level of one or more EVALI-associated biomarker in the subject biological sample as compared to the level of one or more EVALI-associated biomarker in the comparator.

16. The method of claim 15, wherein the substantial difference comprises a statistically significant in the level of one or more EVALI-associated biomarker in the subject biological sample as compared to the level of one or more EVALI-associated biomarker in the comparator.

17. The method of claim 1, wherein said identifying further comprises confirming the presence one or more additional subject-related parameter selected from the group consisting of: EVALI-associated symptoms, EVALI-associated signs, positive screening for vaping or e-cigarette use within the past 90 days, negative screening for common respiratory infections, and chest imaging results indicative of lung injury.

18. The method of claim 17, wherein said EVALI-associated symptoms comprises one or more selected from the group consisting of: cough, chest tightness, dyspnea, shortness of breath, difficulty breathing, sputum production, pleuritic chest pain, fever, malaise, sweats, chills, myalgias, nausea, abdominal pain, emesis, and diarrhea.

19. The method of claim 17, wherein said EVALI-associated signs comprises one or more selected from the group consisting of: hypoxemia, tachypnea, fever, tachycardia, and hypercarbia.

20. The method of claim 1, further comprising treating said subject identified as having EVALI with one or more therapy specific to EVALI.
